(19) United States
(12) Patent Application Publication
(10) Pub. No.: US 2015/0175666 A1
(43) Pub. Date: Jun. 25, 2015

(54) PEPTIDES DERIVED FROM HIV GP41 FOR TREATING T-CELL MEDIATED PATHOLOGIES

(71) Applicant: Yeda Research and Development Co. Ltd., Rehovot (IL)

(72) Inventors: Yechiel Shai, Yahud (IL); Avraham Ashkenazi, Rehovot (IL); Omri Faingold, Rehovot (IL); Avraham Ben Nun, Rehovot (IL); Nathali Kaushansky, Rehovot (IL)

(21) Appl. No.: 14/408,950

(22) PCT Filed: Jun. 25, 2013
(86) PCT No.: PCT/IL2013/050539

§ 371 Date: Dec. 17, 2014

Related U.S. Application Data
(60) Provisional application No. 61/664,778, filed on Jun. 27, 2012.

(51) Int. Cl. C07K 14/005; A61K 38/16; C12N 7/00

(52) U.S. Cl. CPC C07K 14/005; C12N 7/00; A61K 38/162; C12N 2740/16111; C12N 2740/16033

(57) ABSTRACT

The present invention provides peptides, derivatives and analogs comprising an amino acid sequence HTTWMEWD (SEQ ID NO: 1) derived from the ectodomain of HIV gp41 protein, pharmaceutical compositions comprising same, and uses thereof for therapy of T-cell mediated diseases and disorders.

### FIELD OF THE INVENTION

The present invention relates to peptides, derivatives and analogs comprising an amino acid sequence derived from HIV gp41, pharmaceutical compositions comprising same, and uses thereof for therapy of T-cell mediated diseases and disorders.

### BACKGROUND OF THE INVENTION

The immunological synapse (IS) is a complex cellular structure that is formed at the interface of a T-cell and an antigen presenting cell (APC) that expresses the appropriate peptide-major histocompatibility complex (MHC) complexes. It is of the utmost importance and interest due to its involvement in essential steps in the T-cell activation during an immune response. The T-cell receptor (TCR) complex, which resides within the IS, has a critical function in the immune system and serves to protect the organism from infectious agents. The receptor is composed of a heterodimer of TCR-α and β chains which are responsible for antigen recognition. These chains interact with the MHC molecules of the APCs and with the invariant chains of the TCR co-receptor CD3.

Interference with the TCR signal transduction was shown to inhibit T-cell proliferation and activation, leading to compromised immune surveillance. Utilizing this information is crucial for the treatment of autoimmune diseases and other illnesses caused by hyper activation of the immune response. Peptides and lipopeptides, like the transmembrane domain of TCR-α, which interfere with the TCR assembly had already been shown to reduce the clinical signs in several animal models of T-cell mediated (autoimmune) diseases.

International Patent Application No. WO 2007/034490, to an inventor of the present application and co-workers, provides diastereomeric peptides and lipopeptides derived from the T cell receptor alpha (TCRα) transmembrane domain (TMD), pharmaceutical compositions comprising same, and uses thereof for therapy of T cell mediated inflammatory diseases.

International Patent Application No. WO 2007/034489, to one of the inventors of the present application and co-workers, provides immunogenic compositions comprising the T-Cell Receptor constant domain and peptides derived therefrom, effective in preventing or treating T cell mediated inflammatory disease. The immunogenic compositions of WO 2007/034489 comprise at least one immunogen selected from the group consisting of: (i) an isolated constant domain of a chain of a human TCR; and (ii) a peptide comprising an immunogenic fragment of the constant domain of a chain of a TCR.

The Envelope Protein (ENV) of HIV-1

The fusion event of the human immunodeficiency virus-1 (HIV-1) with its target cells is mediated by its envelope glycoprotein (ENV). The ENV is synthesized as a precursor protein, gp160, and undergoes cleavage by cellular proteases into two non-covalently associated subunits, gp120 and gp41. Gp120 is the surface subunit that enables host tropism, while gp41 is the transmembrane subunit responsible for the actual membrane merger.

Gp41 ectodomain comprises several regions (FIG. 1A): the N-terminal fusion peptide (FP), fusion peptide proximal region (FPPR), N-terminal heptad repeat (NHR), disulfide bonded loop, C-terminal heptad repeat (CHR), membrane proximal external region (MPER) and transmembrane domain (TMD). The prevailing models postulate that at least three conformational changes in the gp41 ectodomain are involved in facilitate fusion between the opposing viral and cell membranes (FIG. 1C): (i) a native non-fusogenic state where gp120 subunits shield the hydrophobic gp41 ectodomains; (ii) binding of gp120 to the receptor CD4 and a co-receptor such as CCR5 or CXCR4, causes major conformational changes that drive the transition from the native state into the pre-hairpin intermediate state, Gp41 is released in extended conformation and the FP is inserted into the membrane; (iii) folding of gp41 into a low energy trimeric hairpin conformation which comprises the core structure.

A conserved sequence (named CSK-17) derived from the envelope of several retroviruses was shown to inhibit the proliferation of T cells (Cianciolo, G. J., et al., Science, 1985. 230(4724): p. 453-5). A homologous sequence, identified in the gp41 subunit of HIV-1 ENV, is a 17 mer peptide termed the immunosuppressive unit (ISU) (Ruegg, C. L. et al., J Virol, 1989. 63(8): p. 3257-60). The ISU peptide is derived from the interface between the NHR and the loop regions of gp41 (FIG. 1B) and is capable of inhibiting T-cell proliferation. The ISU is the first immunosuppressive peptide that was designed from the HIV-1 ENV.

International Patent Application No. WO 2006/077601, to an inventor of the present application and co-workers, relates to peptides derived from HIV gp41 fusion peptide (FP) domain useful for prevention or treatment of autoimmune and other T cell mediated pathologies.

International Patent Application No. WO 2011/064770, to an inventor of the present application and co-workers, relates to peptides derived from HIV gp41 transmembrane domain (TMD) useful for therapy of inflammatory diseases or disorders.

The peptides derived from gp41 FP domain and TMD and peptides derived from the transmembrane domain of TCR-α, are membrane inserted peptides and their targets are the TCR or the CD3 that are displayed on the cell membrane surface. Conversely, the ISU peptides acts differently and inhibit the TCR signaling cascade downstream by interfering with calcium influx and the function of protein kinase C (PKC).

International Patent Application No. WO 2005/060350 of one of the inventors of the present invention, discloses membrane binding diastereomeric peptides comprising amino acid sequences corresponding to a fragment of a transmembrane protein, wherein at least two amino acid residues of the diastereomeric peptides being in a D-isomer configuration, useful in inhibiting fusion membrane protein events, including specifically viral replication and transmission. WO 2005/060350 discloses, inter alia, the use of diastereomeric peptides corresponding to amino acids 512 to 544 of HIV-1 gp41 and to amino acids 638 to 673 of HIV-1_{LAV} gp41 for inhibiting membrane fusion processes.

T-Cell Mediated Pathologies

Numerous diseases are believed to result from autoimmune mechanisms. Prominent among these are rheumatoid arthritis, systemic lupus erythematosus, multiple sclerosis, Type I diabetes, myasthenia gravis, pemphigus vulgaris. Autoimmune diseases affect millions of individuals worldwide and the cost of these diseases, in terms of actual treatment expenditures and lost productivity, is measured in billions of dollars annually.

T cells also play a major role in the rejection for organ transplantation or graft versus host disease by bone marrow (hematopoietic stem cell) transplantation. Regulation of such immune responses is therefore therapeutically desired.

A method of treating or inhibiting symptoms of an autoimmune disease by administering a sub-immunogenic amount of an antigen more immunoreactive with alloimmune-immunogen-absorbed (AIA) serum as compared to nonimmune serum of the same species was disclosed in U.S. Pat. No. 5,230,887. One putative antigen, based on its purported serological cross reactivity with MHC Class II antigens, was suggested to be intact gp41 of HIV. The alleged cross reactivity resides in a C-terminal peptide.

Peptides derived form gp41 ectodomain have been described. For instance, U.S. Pat. No. 4,629,783 is related to peptides for the detection of AIDS-related disease and particularly, peptides as reagents in the determination of exposure of a human host to AIDS-related viruses.

U.S. Pat. No. 5,981,706 is directed to methods for synthesizing heat shock protein (HSP)-peptide complexes comprising the steps of adding a shock protein to a denatured protein matrix to bind the HSP to the denatured protein matrix; and adding a complexing solution comprising a peptide to elute a HSP-peptide complex. The '706 publication further provides the HSP-peptide complexes and an apparatus for synthesizing said complexes. Among the peptides suitable for use as complexing agents, according to the '706 publication, are gp41 derived peptides.

U.S. Pat. No. 7,297,784 relates to enhancer peptide sequences originally derived from various retroviral envelope (gp41) protein sequences that enhance the pharmacokinetic properties of any core polypeptide to which they are linked.

Traditional reagents and methods used to regulate an immune response in a patient result in unwanted side effects and limited effectiveness. For example, immunosuppressive reagents (e. g., cyclosporin A, azathioprine, and prednisone) used to treat patients with autoimmune diseases also suppress the patient's entire immune response, thereby increasing the risk of infection, and can cause toxic side effects to non-lymphoid tissues. Due to the medical importance of immune regulation and the inadequacies of existing immunopharmacological reagents, reagents and methods to regulate specific parts of the immune system have been the subject of study for many years.

None of the background art, however, discloses or suggests that peptides derived from the C-terminal heptad repeat (CHR) and CHR proximal region of HIV-1 gp41 ectodomain, regulate T cell activation and in particular are useful in treating or preventing diseases and disorders such as autoimmune diseases, inflammatory diseases and graft rejection.

There exists a long-felt need for more effective means of curing or ameliorating T cell mediated inflammatory or autoimmune diseases and ameliorating T cell mediated pathologies. The development of new immunosuppressive agents capable of selectively inhibiting the activation of T lymphocytes with minimal side effects is therefore desirable.

### SUMMARY OF THE INVENTION

The present invention provides peptides comprising an amino acid sequence derived from the HIV gp41 ectodomain, particularly from the C-terminal heptad repeat (CHR) and CHR proximal region, and pharmaceutical compositions comprising same effective in preventing or treating T cell mediated diseases and disorders, including but not limited to inflammatory diseases, autoimmunity and graft rejection.

The present invention is based, in part, on the unexpected discovery that peptides derived from the CHR and CHR proximal region of gp41 ectodomain inhibit T cell activation. The peptides of the present invention were 20-fold more potent as immunosuppressive peptides as compared to other HIV-derived immunosuppressive peptides (e.g., ISU element). In particular, the peptides inhibited the activation of T-cells specific to the myelin oligodendrocyte glycoprotein (MOG) antigen thereby acting as immunosuppressive compounds in autoimmune diseases including but not limited to multiple sclerosis. Furthermore, said peptides are remarkably less hydrophobic than other HIV-derived immunosuppressive peptides.

Furthermore, the peptides of the invention showed remarkable attenuation of experimental autoimmune encephalomyelitis (EAE) induced in C57BL/6J female mice, whereas demonstrating minimal toxic effect in both in vivo and studies in vitro.

According to one aspect, the present invention provides an isolated peptide of 8-30 amino acids comprising the amino acid sequence HTTWMEWD as set forth in SEQ ID NO: 1, or an analog or a salt thereof. According to another embodiment, said analog comprises a substitution of at least one Trp (W) residue with an amino acid selected from Ile (I), Leu (L) and Gly (G).

According to specific embodiments the isolated peptide comprises an amino acid sequence selected from the group consisting of:

WNHTTWMEWD as set forth in SEQ ID NO: 2,

SNKSLEQIWNHTTWMEWD as set forth in SEQ ID NO: 3, and

EQIWNHTTWMEWDREINN as set forth in SEQ ID NO: 4;

or an analog, a derivative or a salt thereof.

According to another embodiment, said peptide consists of the amino acid sequence WNHTTWMEWD (SEQ ID NO: 2). According to another embodiment, said peptide consists of the amino acid sequence SNKSLEQIWNHTTWMEWD (SEQ ID NO: 3). According to another embodiment, said peptide consists of the amino acid sequence EQIWNHTTWMEWDREINN (SEQ ID NO: 4).

According to another embodiment, the at least one D amino acid is in a position selected from the peptide's N-terminus, C-terminus or both. According to a specific embodiment, the peptide of the present invention consists of the amino acid sequence SNKSLEQIWNHTTWMEWD as set forth in SEQ ID NO: 5, wherein S is D-Ser and D is D-Asp. According to particular embodiments, the peptide comprises or consists of the amino acid sequence as set forth in SEQ ID NO: 11 (WNHTTWMEWD), wherein all amino acids are D-amino acids.

According to another aspect, the present invention provides an isolated peptide of 10-30 amino acids comprising the amino acid sequence WNHTTWMEWD as set forth in SEQ ID NO: 2, or an analog thereof comprising at least one D amino acid.

According to exemplary embodiments, the isolated peptide comprises an amino acid sequence selected from the group consisting of:

SNKSLEQIWNHTTWMEWD (SEQ ID NO: 3),

EQIWNHTTWMEWDREINN (SEQ ID NO: 4), and

SNKSLEQIWNHTTWMEWD, wherein S is D-Ser and D is D-Asp (SEQ ID NO: 5).

According to another aspect, there is provided a pharmaceutical composition comprising as an active ingredient the isolated peptide of the present invention, and a pharmaceutically acceptable carrier.

According to another embodiment, the pharmaceutical composition further comprises an immunosuppressive agent. According to another embodiment, the immunosuppressive agent is an immunosuppressive peptide. According to another embodiment, the immunosuppressive peptide comprises an amino acid sequence LQARILAVERYLKDQQL as set forth in SEQ ID NO: 6, or an analog, derivative or a salt thereof. According to another embodiment, the immunosuppressive peptide consists of an amino acid sequence LQARILAVERYLKDQQL as set forth in SEQ ID NO: 6.

According to another embodiment, there is provided a method of treating a T cell mediated disease or disorder in a subject in need thereof, comprising administering to the subject a therapeutically effective amount of the pharmaceutical composition of the present invention.

According to another embodiment, there is provided use of the pharmaceutical composition of the present invention in the preparation of a medicament for treating a T cell mediated disease or disorder in a subject in need thereof.

According to another embodiment, there is provided a pharmaceutical composition of the present invention for use in treating a T cell mediated disease or disorder in a subject in need thereof.

According to another embodiment, the T cell mediated disease or disorder is a T cell-mediated autoimmune disease. According to another embodiment, the T cell-mediated autoimmune disease is selected from the group consisting of: multiple sclerosis, autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis), autoimmune hepatitis, rheumatoid arthritis, idiopathic thrombocytopenia, scleroderma, alopecia areata, glomerulonephritis, dermatitis and pemphigus. Each possibility represents a separate embodiment of the present invention. According to an exemplary embodiment, the autoimmune disease is multiple sclerosis.

According to another embodiment, the T cell mediated disease or disorder is a T cell-mediated inflammatory disease.

According to another embodiment, the T cell mediated disease or disorder is selected from the group consisting of: allograft rejection and graft-versus-host disease.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A is a schematic representation of the regions within the ectodomain of gp41. Starting from the N-terminus is the fusion peptide (FP), fusion peptide proximal region (FPPR), N-terminal heptad repeat (NHR), disulfide bonded loop, C-terminal heptad repeat (CHR) and the membrane proximal external region (MPER). The sequence (SEQ ID NO: 12) is from HIV-1_{HXB2} and the residue numbers correspond to its gp160 (the boundaries for each region are estimated). The location of the novel motif (SEQ ID NO: 2) is presented in gray and the arrows denote the peptides derived from that region (SEQ ID NOs: 7, 8, 3 and 4).

FIG. 1B shows the amino acid sequence of residues 512-683 of HIV-1_{HXB2} gp160 and the regions noted in FIG. 1A. The sequences of the immunosuppressive unit (ISU) peptide and the immunosuppressive peptide pep 6 (SEQ ID NO: 3) are also indicated.

FIG. 1C illustrates the mechanism of gp41 induced fusion and inhibition. In the native non-fusogenic state gp120 subunits shield the transmembrane gp41. Binding of gp120 to the receptor CD4 and a co-receptor, causes conformational changes releasing gp41 to form an extended pre-hairpin intermediate, characterized by exposed NHR and CHR with the FP inserted into the target membrane. This intermediate is sensitive to the action of fusion inhibitors. In the absence of the inhibitors, gp41 folds into a hairpin conformation followed by hemifusion and formation of a fusion pore (post-fusion).

FIG. 2A depicts the possible C-terminal counterparts of the ISU. (1) is a computational model for the hairpin conformation of HIV-1 gp41 (from PDB 1QCE) including the loop region. (2-4) the location of pep 1 (SEQ ID NO: 7), pep 8 (SEQ ID NO: 8) and pep 6 (SEQ ID NO: 3), respectively, with respect to the ISU.

FIG. 2B shows the bioinformatic analysis for the conservation of pep 6 (SEQ ID NO: 3) within several envelope proteins of retroviral strains. HIV-1, HIV-2, SIV and feline immunodeficiency virus (FIV) are responsible for immunodeficiency phenotype. HTLV-1, HTLV-2, moloney murine leukemia virus (MoLV) and feline leukemia virus (FLV) are responsible for leukemia. (*) resembles conservation of an amino acid between several or all viral strains.

FIG. 2C illustrates the conservation of the motif. The ‘Y’ axis is in bits, which is equal to the relative entropy of the motif in relation to a uniform background frequency model.

FIG. 3A is a bar graph showing inhibition of proliferation by the 41-derived peptides (from left to right: SEQ ID NO: 7, 8, 3 and 4) with increasing concentrations of 2.5 μM, 5 μM, and 10 μM (denoted in gray, black and white, respectively). Results presented are the mean % Inhibition±standard error (SE) of the proliferative response to MOG35-55 peptide (SEQ ID NO: 15) relative to the control (in the absence of the peptides) from a representative experiment (out of four experiments). Uninhibited T-cell proliferative responses were 7866±563 cpm. The background proliferation in the absence of antigen was 132±35 cpm.

FIG. 3B is a line graph depicting dose dependent (0, 2.5, 5, 10 and 20 μM) inhibition of T-cell proliferation by the ISU peptide and its possible counterparts (SEQ ID NO: 1, 3 and 8).

FIG. 3C depicts dose-dependent inhibition of MOG35-55-specific T-cell proliferation of a gp41 derived peptide (SEQ ID NO: 3) starting from the nanomolar concentration range.

FIG. 3D depicts inhibition of T-cell proliferation in the presence or absence of increasing concentrations of the peptide of SEQ ID NO: 3 (▪) or ISU (□), using MOG35-55-specific line T cells cultured with irradiated syngeneic splenocytes as APCs and MOG35-55.

FIG. 3E is a bar graph illustrating the effect of the gp41 derived peptide (SEQ ID NO: 3) on IFNγ secretion by MOG35-55-stimulated T cells (results are mean±SE, n=3. *p<0.05).

FIG. 3F is a bar graph illustrating the effect of the gp41 derived peptide (SEQ ID NO: 3) on TNFα secretion by LPS-stimulated macrophages (results are mean±SE, n=3).

FIG. 3G is a bar graph depicting the viability of Jurkat T cells (column 1) and MOG35-55-specific T cells (column 2) incubated with 20 μM of the gp41 derived peptide (SEQ ID NO:3) for 4 hr. (results are the mean % viability±standard deviation (SD) from the control (cells with no peptide added), n=3).

FIG. 3H is a bar graph depicting the hemolytic activity of the gp41 derived peptide (SEQ ID NO:3). Red blood cells were incubated with 100 μM of the peptide (SEQ ID NO: 3) for 1 hr. (results are mean±SD, n=8. **p<0.01).

FIG. 3I depicts the amino acid sequence of the peptide of SEQ ID NO: 3 as well as the peptides having motif-related mutations SEQ ID NO: 9 and 10. The conserved Trp residues and the conserved acidic residues (Glu and Asp) were mutated to Gly.

FIG. 3J is a bar graph depicting inhibition of MOG35-55-specific T-cell proliferation by peptides of the invention (SEQ ID NOs: 3, 9 and 10 from left to right) with increasing concentrations of 5 μM and 10 μM (denoted in gray and black, respectively). ** p<0.01.

FIGS. 4 A-B show co-localization of the gp-41 derived pep 6 peptide (SEQ ID NO:3) with T-cell membranes in mMOG35-55 T cells (A) and in Jurkat T cells (B) observed utilizing confocal microscopy. The left column is the fluorescence of NBD-pep6 alone. The middle column is the fluorescence of the cytoplasmic or membrane dye. The right column is the merged image between them. Scale bar is equal to 2 μM.

FIG. 4C is a bar graph depicting the percentages of co-localization±SD between pep6 (SEQ ID NO: 3) and cell membrane (black) or and cytoplasm of the cell (gray) in T cells. ** p<0.01.

FIG. 4D is a line graph depicting fluorescent measurements of pep6-membrane interactions.

FIG. 4E depicts binding of the fluorescently labeled peptides (control-antimicrobial not-related peptide; All L peptide—SEQ ID NO: 2; All D peptide—SEQ ID NO: 11) to T-cell lymphocytes derived from mice spleen cells.

FIG. 5A is a bar graph depicting inhibition of MOG35-55-specific T cell proliferation activated in the presence of gp41 derived peptide (SEQ ID NO: 3; 1 μM) by the following agents: (1) MOG35-55 antigen and APCs; (2) CD3 and CD28 antibodies (2 μg/ml) and (3) PMA (50 ng/ml) and ionomycine (1 μM).

FIG. 5B is a biochemical analysis of the interaction of 1 μM fluorescently labeled Rho-pep6 (SEQ ID NO: 3; column 2), Rho-labeled mutant peptide (SEQ ID NO: 9; column 3) and without a peptide (column 1) with T-cell proteins. Actin levels are shown as control.

FIG. 5C is a bar graph showing Rho intensity based on immunoprecipitation of Rho-pep6 with antibodies to TCRα or GFP.

FIGS. 5D-E depict the co-localization of (D) Rho-labeled pep6 (SEQ ID NO: 3) or (E) Rho-labeled control peptide, AMP (SEQ ID NO: 13), with cellular TCRα utilizing confocal microscopy. Scale bar is equal to 2 μM.

FIG. 5F is a bar graph showing the percentage of co-localization±SD between the TCRα and the peptides (pep6 or AMP). ** p<0.01.

FIG. 5G is a line graph showing pep6 (SEQ ID NO: 3) forming α-helical structure in a lipid environment. Circular dichroism spectra of the peptide and its mutant (W/G) (SEQ ID NO: 9), in 1% lysophosphatidylcholine in HEPES.

FIG. 5H depicts fluorescent experiments showing changes in the membrane-bound state of the NBD-labeled core peptide of the TCRαTMD (GLRILLLKV; SEQ ID NO: 14) upon the addition of pep6 (SEQ ID NO: 3) and the control mutant peptides: (SNKSLEQIWNHTTWMGWG; SEQ ID NO: 10) and (SNKSLEQIGNHTTGMEGD; SEQ ID NO: 9) in several sequential doses. Fluorescence spectra were obtained in different NBD-labeled CP/HIV peptide ratios ranging from 40:1 to 5:1 (corresponding to the blue line up to the black line, respectively).

FIG. 6A is a bar graph showing ex vivo down-regulation of encephalitogenic MOG35-55 reactive T cells following administration of a peptide of the invention (SEQ ID NO: 3) to C57Bl mice.

FIGS. 6B-D are bar graphs showing the secretion of IFNγ (B), TNFα (C), and IL-12 (D) pro-inflammatory cytokines from cultured LNCs derived from PBS or pep6 (SEQ ID NO:3)-administered EAE-mice

FIG. 6E is a line graph showing inducement of clinical EAE in mice that were administered PBS (♦) or 0.5 mg/kg of pep6 (SEQ ID NO:3) (▪), n=10 mice per group. Daily rated clinical score is the mean±SE. Results are from a representative experiment (out of two experiments).

FIG. 6F is a bar graph showing ex-vivo down-regulation of encephalitogenic reactive T cells following peptides (All L-SEQ ID NO: 2; All D-SEQ ID NO: 11) administration (0.5 mg/kg). Results are the means±SD. As a control, a non-related peptide was used.

### DETAILED DESCRIPTION OF THE INVENTION

The present invention provides peptides comprising an amino acid sequence derived from the HIV gp41 ectodomain, particularly peptides derived from the CHR and CHR proximal region, and pharmaceutical compositions comprising same effective in preventing or treating T cell mediated diseases and disorders, including but not limited to inflammatory diseases, autoimmunity and graft rejection.

The CHR region resides approximately between amino acids 629-661 of gp160 HIV-1_{HXB2} strain (GenBank accession number K03455). The “CHR proximal region” as used herein, refers to at least 3 amino acids contiguous to CHR's amino terminus, i.e., within the loop region. Peptides derived from the CHR and CHR proximal region, in some embodiments, comprise an amino acid sequence corresponding to amino acid 615-637 of gp160, or an analog or a fragment thereof.

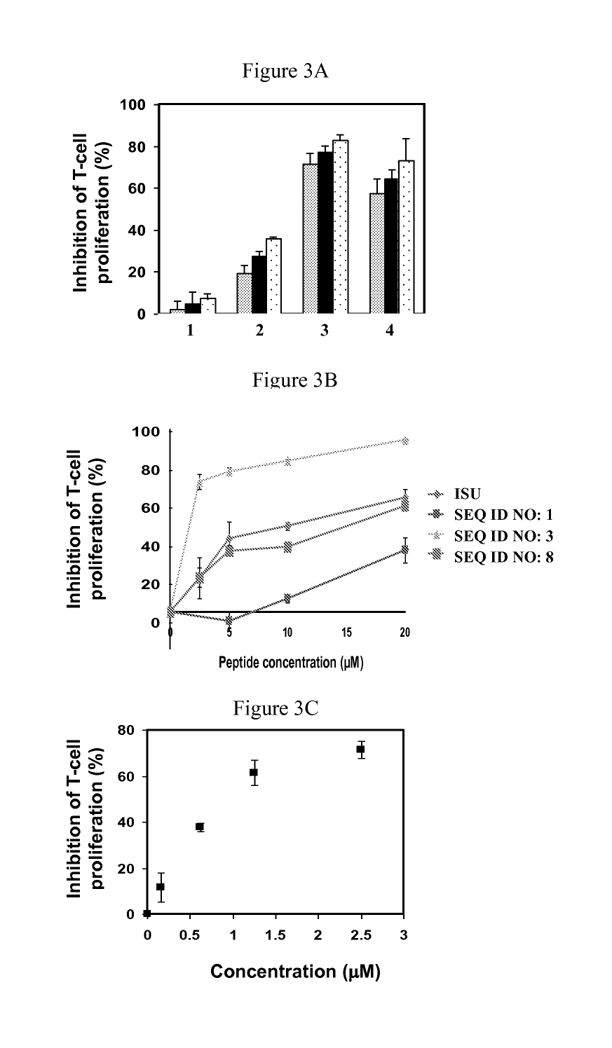

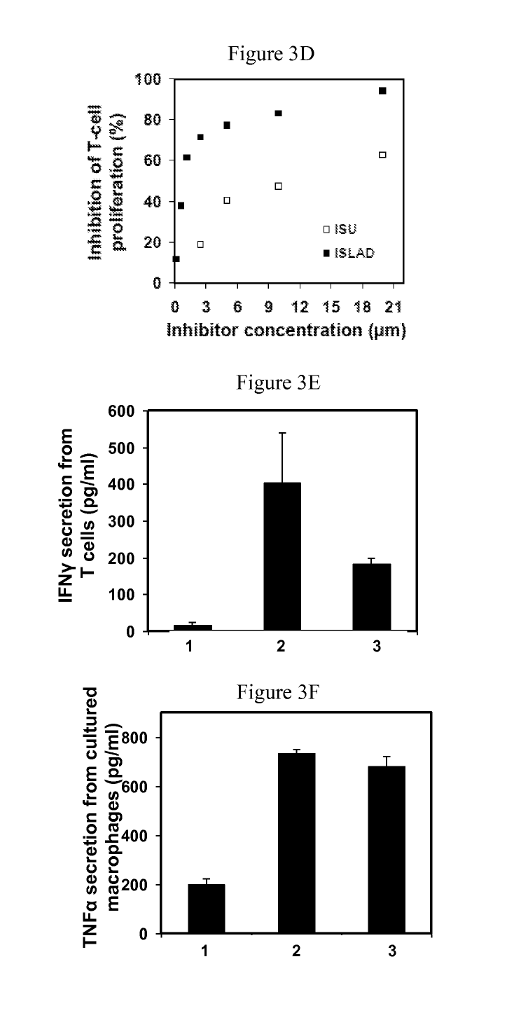

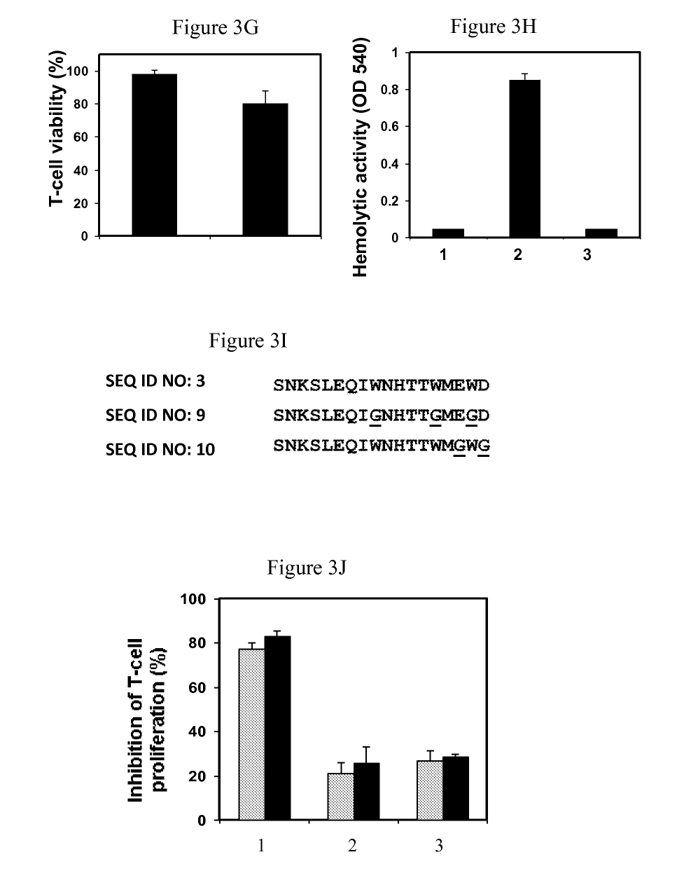

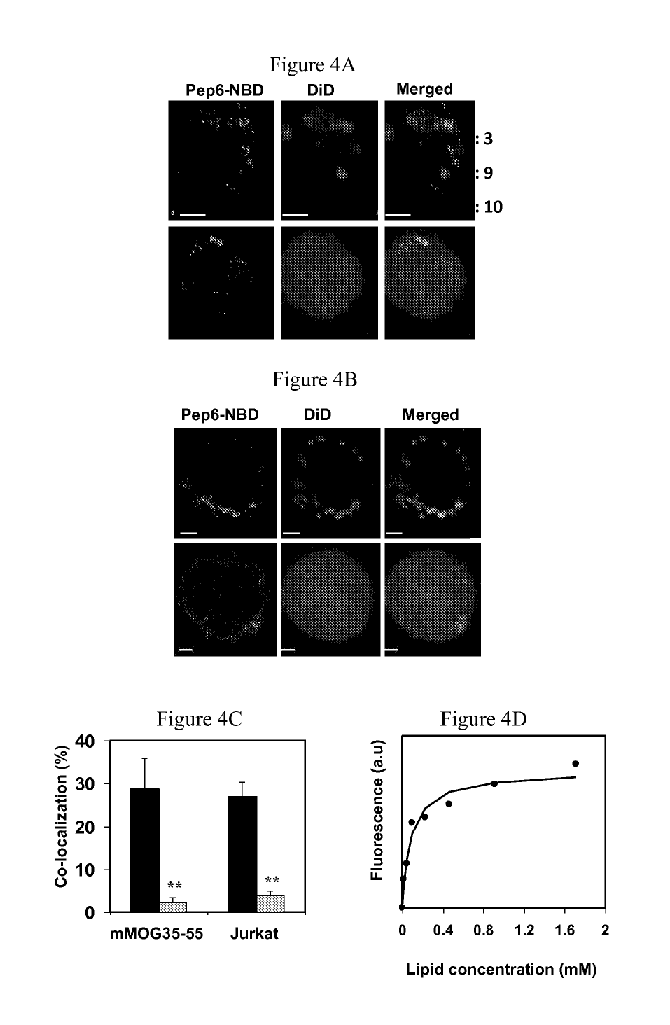

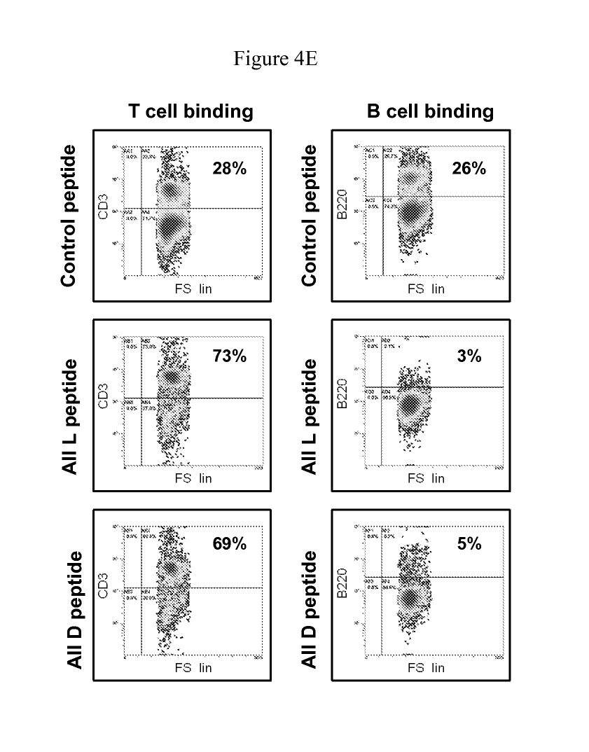

As exemplified herein below, a novel conserved element (SEQ ID NO: 2; also referred herein as motif) derived from the gp41 subunit of HIV ENV, particularly from the CHR and CHR proximal region, is able to inhibit T-cells activation. The immunosuppressive effects exerted by the peptides of the invention are supported and disclosed in Example 2 herein. As exemplified herein, the peptides inhibit the activation and proliferation of T-cells specific to the MOG (myelin oligodendrocyte glycoprotein) antigen (FIGS. 3A and 4) as well as pro-inflammatory cytokine release. The results disclosed herein indicate the peptides' role as immunosuppressive compounds in autoimmune diseases such as multiple sclerosis in which the T cells recognize myelin as foreign and attack it. Further, EAE induced mice treated with a peptide of the invention showed remarkable attenuation of the disease (Example 5).

While it was previously reported that different peptides from HIV-1 gp41 are able to suppress T-cell activation, the peptides of the invention exhibit unique and advantageous properties. The peptides are about 20 times more potent then the strongest peptide reported from HIV-1 ENV (Table 1). Further, the peptides exhibit minimal toxicity to cells. As exemplified in Example 2, at the highest concentration tested (50 times its IC₅₀), the peptides were still not toxic (FIG. 3G). Furthermore, the peptides of the invention are less hydrophobic than other gp41 derived immunosuppressive peptides (e.g., the FP and the TMD of gp41), and therefore, are more soluble in aqueous solution. Further, said peptides interact with another immunosuppressive element, the ISU, indicating a synergistic inhibitory effect.

In some embodiments, there is provided an isolated peptide of 8-30 amino comprising the amino acid sequence HTTWMEWD (SEQ ID NO: 1), or an analog, derivative or a salt thereof. In specific embodiments the peptides of the invention are selected from the group consisting of: WNHTTWMEWD (SEQ ID NO: 2), SNKSLEQIWNHTTWMEWD (SEQ ID NO: 3), EQIWNHTTWMEWDREINN (SEQ ID NO: 4), or an analog, derivative or a salt thereof.

The term “peptide” as used herein encompasses native peptides (degradation products, synthetic peptides or recombinant peptides), peptidomimetics (typically including non peptide bonds or other synthetic modifications) and the peptide analogues peptoids and semipeptoids, and may have, for example, modifications rendering the peptides more stable while in the body or more capable of penetrating into cells. Peptides typically consist of a sequence of about 3 to about 50 amino acids. According to a particular embodiment, the peptides of the present invention consist of 6-50 amino acids, 7-45 amino acids, 8-40 amino acids, 8-35 amino acids, 8-30 amino acids, 10-30 amino acids, 10-28 amino acids, 10-26 amino acids, 10-24 amino acids, 10-22 amino acids, 10-20 amino acids or 10-18 amino acids, wherein each possibility represents a separate embodiment of the present invention.

According to another embodiment, the peptides of the present invention consist of at least 8 amino acids, at least 9 amino acids or at least 10 amino acids wherein each possibility represents a separate embodiment of the present invention.

According to yet another embodiment, the peptides of the present invention consists of at most 50, at most 45, at most 40, at most 35, at most 30, at most 28 amino acids, at most 26 amino acids, at most 25 amino acids, at most 24 amino acids, at most 23 amino acids, at most 22 amino acids, at most 21 amino acids, at most 20 amino acids, at most 19 amino acids, at most 18 amino acids, wherein each possibility represents a separate embodiment of the present invention

The terms “polypeptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “isolated” peptide refers to a peptide that is essentially free from contaminating cellular components, such as carbohydrate, lipid, or other proteinaceous impurities associated with the peptide in nature. Typically, a preparation of isolated peptide contains the peptide in a highly purified form, i.e., at least about 80% pure, at least about 90% pure, at least about 95% pure, greater than 95% pure, or greater than 99% pure.

The present invention further provides fragments, analogs and chemical modifications of the peptides of the present invention as long as they are capable of modulating (e.g. reducing or inhibiting) T cell activity.

In another embodiment, said analog comprises a substitution of at least one Trp (W) residue with an amino acid selected from Ile (I), Leu (L) and Gly (G). In yet another embodiment, said analog comprises a substitution of at least one Trp (W) residue with an amino acid selected from Ile (I) and Leu (L).

According to a specific embodiment, said substitution is at a residue selected from the group consisting of the residues in position 3-8 of SEQ ID NO: 1. According to another specific embodiment, said substitution is at a residue selected from the group consisting of the residues in position 1, 2, 5-10 of SEQ ID NO: 2.

According to some embodiments, the analog of the peptide of the invention comprises 1-3 amino acid modifications relative to SEQ ID NO: 1, wherein each amino acid modification is independently selected from the group consisting of a substitution, an insertion and a deletion. According to some embodiments, the analog of the peptide of the invention comprises 1-3 amino acid modifications relative to SEQ ID NO: 2, wherein each amino acid modification is independently selected from the group consisting of a substitution, an insertion and a deletion.

According to a specific embodiment, the insertion is of a charged amino acid. According to another specific embodiment, the substitution is to a charged amino acid. Amino acids with positively charged side chains (basic amino acids) include Arginine (R), Lysine (K) and Histidine (H). Amino acids with negatively charged side chains include Aspartic acid (D) and Glutamic acid (E).

According to a particular embodiment, said substitution is a conservative substitution. One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to aspartic acid (D). Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

6) Phenylalanine (F), Tyrosine (Y), Tryptophan (W) (see, e.g., Creighton, Proteins, 1984).

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

The phrase “conservative substitution” also includes the use of a chemically derivatized residue in place of a non-derivatized residue provided that such peptide displays the requisite function of modulating the immune system's innate response as specified herein.

The term “derived from” or “corresponding to” refers to construction of a peptide based on the knowledge of a sequence using any one of the suitable means known to one skilled in the art, e.g. chemical synthesis in accordance with standard protocols in the art. A peptide derived from, or corresponding to amino acid 615-637 of HIV gp160 (gp41 CHR and CHR proximal region) can be an analog, fragment, conjugate or derivative of a native amino acid 615-637 of gp160, and salts thereof, as long as said peptide retains its ability to inhibit T cell activation.

According to specific embodiments, the peptide of the invention does not comprise an Asn residue as a substitute of the His residue in position 3 of SEQ ID NO: 2. According to another embodiment, the analog does not comprise a Met residue as a substitute of the Thr residue in position 4 of SEQ ID NO: 2. According to another embodiment, said analog does not comprise Asn-Met as a substitute of His-Thr in position 3-4 of SEQ ID NO: 2.

According to another embodiment, said analog has at least 75% sequence identity to SEQ ID NO: 1. According to another embodiment, said analog has at least 85% sequence identity to SEQ ID NO: 1.

According to another embodiment, said analog has at least 70% sequence identity to SEQ ID NO: 2. According to another embodiment, said analog has at least 80% sequence identity to SEQ ID NO: 2. According to another embodiment, said analog has at least 90% sequence identity to SEQ ID NO: 3.

Percentage sequence identity can be determined, for example, by the Fitch et al. version of the algorithm (Fitch et al, Proc. Natl. Acad. Sci. U.S.A. 80: 1382-1386 (1983)) described by Needleman et al, (Needleman et al, J. MoI. Biol. 48: 443-453 (1970)), after aligning the sequences to provide for maximum homology. Alternatively, the determination of percent identity between two sequences can be accomplished using the mathematical algorithm of Karlin and Altschul (1993) Proc. Natl. Acad. Sci. USA 90:5873-5877. Such an algorithm is incorporated into the BLASTP program of Altschul et al. (1990) J. MoI. Biol. 215, 403-410. BLAST protein searches are performed with the BLASTP program to obtain amino acid sequences homologous to SEQ ID NO: 4. To obtain gapped alignments for comparative purposes, Gapped BLAST is utilized as described in Altschul et al (1997) Nucleic Acids Res. 25:3389-3402. When utilizing BLAST and Gapped BLAST programs, the default parameters of the respective programs (e.g., XBLAST) are used.

Typically, the present invention encompasses derivatives of the peptides. The term “derivative” or “chemical derivative” includes any chemical derivative of the peptide having one or more residues chemically derivatized by reaction of side chains or functional groups. Such derivatized molecules include, for example, those molecules in which free amino groups have been derivatized. Examples of amine derivatives include amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters, amines or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a peptide derivative can differ from the natural sequence of the peptides of the invention by chemical modifications including, but are not limited to, terminal-NH₂ acylation, acetylation, or thioglycolic acid amidation, and by terminal-carboxlyamidation, e.g., with ammonia, methylamine, and the like. Peptides can be either linear, cyclic or branched and the like, which conformations can be achieved using methods well known in the art.

The peptide derivatives and analogs according to the principles of the present invention can also include side chain bond modifications, including but not limited to —CH₂—NH—, —CH₂—S—, —CH₂—S═O, O═C—NH—, —CH₂—O—, —CH₂—CH₂—, S═C—NH—, and —CH═CH—, and backbone modifications such as modified peptide bonds. Peptide bonds (—CO—NH—) within the peptide can be substituted, for example, by N-methylated bonds (—N(CH3)—CO—); ester bonds (—C(R)H—C—O—O—C(R)H—N); ketomethylene bonds (—CO—CH2-); α-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH); olefinic double bonds (—CH═CH—); and peptide derivatives (—N(R)—CH2-CO—), wherein R is the “normal” side chain, naturally presented on the carbon atom. These modifications can occur at one or more of the bonds along the peptide chain and even at several (e.g., 2-3) at the same time.

The present invention also encompasses peptide derivatives and analogs in which free amino groups have been derivatized to form amine hydrochlorides, p-toluene sulfonylamino groups, carbobenzoxyamino groups, t-butyloxycarbonylamino groups, chloroacetylamino groups or formylamino groups. Free carboxyl groups may be derivatized to form, for example, salts, methyl and ethyl esters or other types of esters, amines or hydrazides. The imidazole nitrogen of histidine can be derivatized to form N-im-benzylhistidine.

The peptide analogs can also contain non-natural amino acids. Examples of non-natural amino acids include, but are not limited to, sarcosine (Sar), norleucine, ornithine, citrulline, diaminobutyric acid, homoserine, isopropyl Lys, 3-(2′-naphtyl)-Ala, nicotinyl Lys, amino isobutyric acid, and 3-(3′-pyridyl-Ala).

Furthermore, the peptide analogs can contain other derivatized amino acid residues including, but not limited to, methylated amino acids, N-benzylated amino acids, O-benzylated amino acids, N-acetylated amino acids, O-acetylated amino acids, carbobenzoxy-substituted amino acids and the like. Specific examples include, but are not limited to, methyl-Ala (MeAla), MeTyr, MeArg, MeGlu, MeVal, MeHis, N-acetyl-Lys, O-acetyl-Lys, carbobenzoxy-Lys, Tyr-O-Benzyl, Glu-O-Benzyl, Benzyl-His, Arg-Tosyl, t-butylglycine, t-butylalanine, phenylglycine, cyclohexylalanine, and the like.

The term “derivative” may further include chemical derivatives of the hydrophobic (e.g., fatty acid) moieties.

The invention further includes peptide analogs, which can contain one or more D-isomer forms of the amino acids. Production of retro-inverso D-amino acid peptides where at least one amino acid, and perhaps all amino acids are D-amino acids is well known in the art. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein. In a particular embodiment, all amino acids of the peptides of the invention are D-amino acids.

According to a particular embodiment, the peptides of the present invention are diastereomeric peptides. The diastereomeric peptides are highly advantageous over all L- or all D-amino acid peptides having the same amino acid sequence because of their higher water solubility, lower immunogenicity (see, for example, Benkirane, N., et al., 1993, J. Biol. Chem. 268: 26279-26285), and lower susceptibility to proteolytic degradation. Such characteristics endow the diastereomeric peptides with higher efficacy and higher bioavailability than those of the all L or all D-amino acid peptides comprising the same amino acid sequence.

The term “diastereomeric peptide” as used herein refers to a peptide comprising both L-amino acid residues and D-amino acid residues. The number and position of D-amino acid residues in a diastereomeric peptide of the preset invention may be variable so long as the peptides are capable on modulating the immune system's innate response. In some embodiments, the peptides comprises at least 2 D-amino acid residues, at least 3 D-amino acid residues, at least 4 D-amino acid residues, at least 5 D-amino acid residues, at least 6 D-amino acid residues, at least 7 D-amino acid residues, at least 8 D-amino acid residues, at least 9 D-amino acid residues, wherein each possibility represents a separate embodiment of the invention.

According to another embodiment, the at least one D amino acid is in a position selected from the peptide's N-terminus, C-terminus or both. According to a specific embodiment, the peptide of diastereomeric peptide consists of the amino acid sequence SNKSLEQIWNHTTWMEWD as set forth in SEQ ID NO: 5, wherein S is D-Ser and D is D-Asp

The peptides of the invention may be synthesized or prepared by techniques well known in the art. The peptides can be synthesized by a solid phase peptide synthesis method of Merrifield (see J. Am. Chem. Soc., 85:2149, 1964). Alternatively, the peptides of the present invention can be synthesized using standard solution methods well known in the art (see, for example, Bodanszky, M., Principles of Peptide Synthesis, Springer-Verlag, 1984) or by any other method known in the art for peptide synthesis.

In general, these methods comprise sequential addition of one or more amino acids or suitably protected amino acids to a growing peptide chain bound to a suitable resin.

Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha,alpha)-dimethyl-3,5 dimethoxybenzyloxycarbonyl, o-nitrophenylsulfenyl, 2-cyano-t-butyloxycarbonyl, 9-fluorenylmethyloxycarbonyl (FMOC) and the like. The BOC or FMOC protecting group is preferred.

In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

The peptides of the invention may alternatively be synthesized such that one or more of the bonds, which link the amino acid residues of the peptides are non-peptide bonds. These alternative non-peptide bonds include, but are not limited to, imino, ester, hydrazide, semicarbazide, and azo bonds, which can be formed by reactions well known to skilled in the art.

The peptides of the present invention, analogs, or derivatives thereof produced by recombinant techniques can be purified so that the peptides will be substantially pure when administered to a subject. The term “substantially pure” refers to a compound, e.g., a peptide, which has been separated from components, which naturally accompany it. Typically, a peptide is substantially pure when at least 50%, preferably at least 75%, more preferably at least 90%, and most preferably at least 99% of the total material (by volume, by wet or dry weight, or by mole percent or mole fraction) in a sample is the peptide of interest. Purity can be measured by any appropriate method, e.g., in the case of peptides by HPLC analysis.

Included within the scope of the invention are peptide conjugates comprising the peptides of the present invention derivatives, or analogs thereof joined at their amino or carboxy-terminus or at one of the side chains via a peptide bond to an amino acid sequence of a different protein. Additionally or alternatively, the peptides of the present invention, derivatives, or analogs thereof can be joined to another moiety such as, for example, a fatty acid, a sugar moiety, arginine residues, hydrophobic moieties, and any known moiety that facilitate membrane or cell penetration. Conjugates comprising peptides of the invention and a protein can be made by protein synthesis, e. g., by use of a peptide synthesizer, or by ligating the appropriate nucleic acid sequences encoding the desired amino acid sequences to each other by methods known in the art, in the proper coding frame, and expressing the conjugate by methods commonly known in the art.

Addition of amino acid residues may be performed at either terminus of the peptides of the invention for the purpose of providing a “linker” by which the peptides of this invention can be conveniently bound to a carrier. Such linkers are usually of at least one amino acid residue and can be of 40 or more residues, more often of 1 to 10 residues. Typical amino acid residues used for linking are tyrosine, cysteine, lysine, glutamic and aspartic acid, or the like.

According to another aspect, the present invention provides an isolated polynucleotide sequence encoding the peptides of the present invention, or an analog or a conjugate thereof, the peptides of the present invention, analog or conjugate thereof inhibits T cell activity, thereby useful for treating T cell mediated diseases and disorders.

The term “polynucleotide” means a polymer of deoxyribonucleic acid (DNA), ribonucleic acid (RNA) or a combination thereof, which can be derived from any source, can be single- or double-stranded, and can optionally contain synthetic, non-natural, or altered nucleotides, which are capable of being incorporated into DNA or RNA polymers.

An “isolated polynucleotide” refers to a polynucleotide segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to polynucleotides, which have been substantially purified from other components, which naturally accompany the polynucleotide in the cell, e.g., RNA or DNA or proteins. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence, and RNA such as mRNA.

The term “encoding” refers to the inherent property of specific sequences of nucleotides in an isolated polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a peptide or protein if transcription and translation of mRNA corresponding to that gene produces the peptide or protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the peptide or protein or other product of that gene or cDNA.

One who is skilled in the art will appreciate that more than one polynucleotide may encode any given peptide or protein in view of the degeneracy of the genetic code and the allowance of exceptions to classical base pairing in the third position of the codon, as given by the so-called “Wobble rules.” It is intended that the present invention encompass polynucleotides that encode the peptides of the present invention as well as any analog thereof.

A polynucleotide of the present invention can be expressed as a secreted peptide where the peptides of the present invention or analog thereof is isolated from the medium in which the host cell containing the polynucleotide is grown, or the polynucleotide can be expressed as an intracellular peptide by deleting the leader or other peptides, in which case the peptides of the present invention or analog thereof is isolated from the host cells. The peptides of the present invention or analog thereof are then purified by standard protein purification methods known in the art.

The peptides of the present invention, analogs, or derivatives thereof can also be provided to the tissue of interest by transferring an expression vector comprising an isolated polynucleotide encoding the peptides of the present invention, or analog thereof to cells associated with the tissue of interest. The cells produce the peptide such that it is suitably provided to the cells within the tissue to exert a biological activity such as, for example, to reduce or inhibit inflammatory processes within the tissue of interest.

The expression vector according to the principles of the present invention further comprises a promoter. In the context of the present invention, the promoter must be able to drive the expression of the peptide within the cells. Many viral promoters are appropriate for use in such an expression vector (e.g., retroviral ITRs, LTRs, immediate early viral promoters (IEp) (such as herpes virus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters). Other suitable promoters are eukaryotic promoters, which contain enhancer sequences (e.g., the rabbit β-globin regulatory elements), constitutively active promoters (e.g., the β-actin promoter, etc.), signal and/or tissue specific promoters (e.g., inducible and/or repressible promoters, such as a promoter responsive to TNF or RU486, the metallothionine promoter, etc.), and tumor-specific promoters.

Within the expression vector, the polynucleotide encoding the peptides of the present invention, or analog thereof and the promoter are operably linked such that the promoter is able to drive the expression of the polynucleotide. As long as this operable linkage is maintained, the expression vector can include more than one gene, such as multiple genes separated by internal ribosome entry sites (IRES). Furthermore, the expression vector can optionally include other elements, such as splice sites, polyadenylation sequences, transcriptional regulatory elements (e.g., enhancers, silencers, etc.), or other sequences.

The expression vectors are introduced into the cells in a manner such that they are capable of expressing the isolated polynucleotide encoding the peptides of the present invention or analog thereof contained therein. Any suitable vector can be so employed, many of which are known in the art. Examples of such vectors include naked DNA vectors (such as oligonucleotides or plasmids), viral vectors such as adeno-associated viral vectors (Berns et al., 1995, Ann. N.Y. Acad. Sci. 772:95-104, the contents of which are hereby incorporated by reference in their entirety), adenoviral vectors, herpes virus vectors (Fink et al., 1996, Ann. Rev. Neurosci. 19:265-287), packaged amplicons (Federoff et al., 1992, Proc. Natl. Acad. Sci. USA 89:1636-1640, the contents of which are hereby incorporated by reference in their entirety), papilloma virus vectors, picornavirus vectors, polyoma virus vectors, retroviral vectors, SV40 viral vectors, vaccinia virus vectors, and other vectors. Additionally, the vector can also include other genetic elements, such as, for example, genes encoding a selectable marker (e.g., β-gal or a marker conferring resistance to a toxin), a pharmacologically active protein, a transcription factor, or other biologically active substance.

Methods for manipulating a vector comprising an isolated polynucleotide are well known in the art (e.g., Sambrook et al., 1989, Molecular Cloning: A Laboratory Manual, 2d edition, Cold Spring Harbor Press, the contents of which are hereby incorporated by reference in their entirety) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The expression vector comprising the polynucleotide of interest is introduced into the cells by any means appropriate for the transfer of DNA into cells. Many such methods are well known in the art (e.g., Sambrook et al., supra; see also Watson et al., 1992, Recombinant DNA, Chapter 12, 2d edition, Scientific American Books, the contents of which are hereby incorporated by reference in their entirety). Thus, in the case of prokaryotic cells, vector introduction can be accomplished, for example, by electroporation, transformation, transduction, conjugation, or mobilization. For eukaryotic cells, vectors can be introduced through the use of, for example, electroporation, transfection, infection, DNA coated microprojectiles, or protoplast fusion. Examples of eukaryotic cells into which the expression vector can be introduced include, but are not limited to, ovum, stem cells, blastocytes, and the like.

Cells, into which the polynucleotide has been transferred under the control of an inducible promoter if necessary, can be used as transient transformants. Such cells themselves may then be transferred into a subject for therapeutic benefit therein. Thus, the cells can be transferred to a site in the subject such that the peptide of the invention is expressed therein and secreted therefrom and thus reduces or inhibits, for example, T cell mediated processes so that the clinical condition of the subject is improved. Alternatively, particularly in the case of cells to which the vector has been added in vitro, the cells can first be subjected to several rounds of clonal selection (facilitated usually by the use of a selectable marker sequence in the vector) to select for stable transformants. Such stable transformants are then transferred to a subject, preferably a human, for therapeutic benefit therein.

Within the cells, the polynucleotide encoding the peptides of the present invention, or analog thereof is expressed, and optionally is secreted. Successful expression of the polynucleotide can be assessed using standard molecular biology techniques (e.g., Northern hybridization, Western blotting, immunoprecipitation, enzyme immunoassay, etc.).

The present invention encompasses transgenic animals comprising an isolated polynucleotide encoding the peptides of the invention.

### Pharmaceutical Compositions

The present invention provides pharmaceutical compositions comprising as an active ingredient a therapeutically effective amount of a peptide of the present invention, and a pharmaceutically acceptable carrier.

The pharmaceutical compositions of the invention can be formulated in the form of a pharmaceutically acceptable salt of the peptides of the present invention or their analogs or derivatives thereof. Pharmaceutically acceptable salts include those salts formed with free amino groups such as salts derived from non-toxic inorganic or organic acids such as hydrochloric, phosphoric, acetic, oxalic, tartaric acids, and the like, and those salts formed with free carboxyl groups such as salts derived from non-toxic inorganic or organic bases such as sodium, potassium, ammonium, calcium, ferric hydroxides, isopropylamine, triethylamine, 2-ethylamino ethanol, histidine, procaine, and the like.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned.

The compositions can take the form of solutions, suspensions, emulsions, tablets, pills, capsules, powders, gels, creams, ointments, foams, pastes, sustained-release formulations and the like. The compositions can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences by E.W. Martin, the contents of which are hereby incorporated by reference herein. Such compositions will contain a therapeutically effective amount of a gp41 derived peptide (i.e. comprising the motif of SEQ ID NO: 1) according to the present invention, preferably in a substantially purified form, together with a suitable amount of carrier so as to provide the form for proper administration to the subject.

The amount of the peptides of the present invention, which will be effective in the treatment of a particular disorder or condition will depend on the nature of the disorder or condition and on the particular peptide and can be determined by standard clinical techniques known to a person skilled in the art. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

It will be understood that the pharmaceutical compositions of the present invention can comprise the peptides of the current invention, or a derivative or analog thereof, or all possible combinations of two or more of these peptide derivatives, or analogs or other sources of the peptide of the current invention. Thus, the pharmaceutical compositions can comprise one or more isolated polynucleotides, one or more expression vectors, or one or more host cells or any combination thereof, according to the principles of the present invention.

Determination of a therapeutically effective amount of a peptide is well within the capability of those skilled in the art, especially in light of the detailed disclosure provided herein.

Toxicity and therapeutic efficacy of the peptides described herein can be determined by standard pharmaceutical procedures in cell cultures or experimental animals, e.g., by determining the IC₅₀ (the concentration which provides 50% inhibition) and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See, for example, Fingl et al., 1975, in The Pharmacological Basis of Therapeutics, Ch. 1 p. 1, the contents of which are hereby incorporated by reference in their entirety).

Depending on the severity and responsiveness of the condition to be treated, dosing can also be a single administration of a slow release composition, with course of treatment lasting from several days to several weeks or until cure is effected or diminution of the disease state is achieved.

It is further understood that the peptides of the invention can be formulated or administered together with additional active ingredients as required to treat the condition of the patient. In a specific embodiment, the pharmaceutical composition further comprises an immunosuppressive agent. According to another embodiment, the immunosuppressive agent is an immunosuppressive peptide. According to another embodiment, the immunosuppressive peptide comprises an amino acid sequence LQARILAVERYLKDQQL as set forth in SEQ ID NO: 6, or an analog, derivative or a salt thereof. According to another embodiment, the immunosuppressive peptide consists of an amino acid sequence LQARILAVERYLKDQQL as set forth in SEQ ID NO: 6.

Depending on the location of the tissue of interest, the peptides of the present invention can be supplied in any manner suitable for the provision of the peptide to cells within the tissue of interest. Thus, for example, a composition containing the peptides of the present invention can be introduced, for example, into the systemic circulation, which will distribute the peptide to the tissue of interest. Alternatively, a composition can be applied topically to the tissue of interest (e.g., injected, or pumped as a continuous infusion, or as a bolus within a tissue, applied to all or a portion of the surface of the skin, etc.).

The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

It may be desirable to administer the pharmaceutical composition of the invention locally to the area in need of treatment; this can be achieved by, for example, and not by way of limitation, local infusion, topical application, by injection, by means of a catheter, by means of a suppository, or by means of an implant, said implant being of a porous, non-porous, or gelatinous material. According to some preferred embodiments, administration can be by direct injection e.g., via a syringe, at the site of a damaged tissue.

For topical application, a peptide of the present invention, derivative, or analog thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as a fatty oil. In addition, dosage unit forms can contain various other materials which modify the physical form of the dosage unit, for example, coatings of sugar, shellac, or other enteric agents. The tablets of the invention can further be film coated.

The peptides of the present invention, derivatives or analogs thereof can be delivered in a controlled release system. Thus, an infusion pump can be used to administer the peptide such as the one that is used, for example, for delivering insulin or chemotherapy to specific organs or tumors. In one embodiment, the peptide of the invention is administered in combination with a biodegradable, biocompatible polymeric implant, which releases the peptide over a controlled period of time at a selected site. Examples of preferred polymeric materials include, but are not limited to, polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla., the contents of which are hereby incorporated by reference in their entirety). In yet another embodiment, a controlled release system can be placed in proximity to a therapeutic target, thus requiring only a fraction of the systemic dose.

Use of the Peptides

In some aspects, the present invention provides the use of pharmaceutical compositions comprising the immunosuppressive peptides of the invention, effective in preventing or treating T cell mediated diseases and disorders.

T lymphocytes (T cells) are one of a variety of distinct cell types involved in an immune response. The activity of T cells is regulated by antigen, presented to a T cell, by an antigen-presenting cell (APC), in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell.

Proper activation of T lymphocytes by antigen-presenting cells requires stimulation not only of the TCR, but the combined and coordinated engagement of its co-receptors. Most TCR co-receptors bind cell-surface ligands and are concentrated in areas of cell-cell contact, forming what has been termed an immune synapse. Synapse formation has been associated with the induction of antigen-specific T cell proliferation, cytokine production and lytic granule release, and its function was determined necessary for complete T cell activation.

Thus according to some aspects, the present invention provides a method for treating a T cell mediated disease or disorder. The terms “T cell mediated disease or disorder” and “T-cell mediated pathology” refer to any condition in which an inappropriate or detrimental T cell response is a component of the etiology or pathology of a disease or disorder. The term is intended to include both diseases directly mediated by T cells, and also diseases in which an inappropriate or detrimental T cell response contributes to the production of abnormal antibodies, as well as graft rejection. It should be understood that the term does not include diseases or conditions caused by HIV, such as acquired immune deficiency syndrome (AIDS).

In one embodiment of the invention, the peptides and compositions are useful for treating a T cell-mediated autoimmune disease, including but not limited to: multiple sclerosis (MS), autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis) or autoimmune hepatitis, rheumatoid arthritis. Each possibility represents a separate embodiment of the present invention.

In other embodiments the composition is useful for treating a T cell-mediated inflammatory disease, including but not limited to: inflammatory or allergic diseases such as asthma, hypersensitivity lung diseases, hypersensitivity pneumonitis, delayed-type hypersensitivity, interstitial lung disease (ILD) (e.g., idiopathic pulmonary fibrosis, or ILD associated with rheumatoid arthritis or other inflammatory diseases); scleroderma; psoriasis (including T-cell mediated psoriasis); dermatitis (including atopic dermatitis and eczematous dermatitis), iritis, conjunctivitis, keratoconjunctivitis, cutaneous lupus erythematosus, scleroderma, vaginitis, proctitis, drug eruptions, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Graves ophthalmopathy and primary biliary cirrhosis. Each possibility represents a separate embodiment of the present invention.

In other embodiments, the composition is useful for treating graft rejection, including allograft rejection or graft-versus-host disease.

In additional embodiments, the peptides and compositions are capable of inhibiting or reducing pro-inflammatory cytokine release (e.g., IFNγ).

In another aspect, the invention is directed to the use of an isolated peptide of the present invention, for the preparation of a pharmaceutical composition for treating or preventing T cell mediated disease and disorders.

In another aspect, the invention is directed to an isolated peptide according to the present invention, for use in treating or preventing T cell mediated disease and disorders.

Methods of treating a disease according to the invention may include administration of the pharmaceutical compositions of the present invention as a single active agent, or in combination with additional methods of treatment. The methods of treatment of the invention may be in parallel to, prior to, or following additional methods of treatment.

The following examples are presented in order to more fully illustrate some embodiments of the invention. They should, in no way be construed, however, as limiting the broad scope of the invention.

### EXAMPLES

### Peptide Synthesis

Peptides were synthesized on Rink Amide MBHA resin by using the F-moc strategy (amino acids from Calibochem-Novabiochem AG, Switzerland). Peptides were cleaved from the resin by a TFA: DDW: TES (93.1:4.9:2 (v/v)) mixture, and purified by reverse phase high performance liquid chromatography (RP-HPLC) to >95% homogeneity. The molecular weight of the peptides was confirmed by platform LCA electrospray mass spectrometry. Addition of 4-chloro-7-nitrobenz-2-oxa-1,3-diazole fluoride (NBD-F) (Molecular Probes, USA) to the N-terminus of selected peptides was performed in dimethylformamide (DMF) for 1 hour. Addition of rhodamine-N-hydroxysuccinimide (Rho) (Molecular Probes, USA) to N-terminus of selected peptides was performed in DMF containing 2% N,NDiisopropylethylamine (DIEA) for 24 hours.

Cell Lines

Antigen-specific T-cell lines were selected in vitro from primed lymph node cells derived from C57Bl/6J mice that had been immunized 9 days before with antigen (100 μg myelin peptide, MOG35-55) emulsified in CFA containing 150 μg Mycobacterium tuberculosis (Mt) H37Ra (Difco Laboratories, Detroit, Mich.). All T-cell lines were maintained in vitro in medium containing IL-2 with alternate stimulation with the antigen, every 10-14 days. The human T-cell-line, Jurkat E6-1, was obtained through the AIDS Research and Reference Reagent Program, Division of AIDS, NIAID, NIH. RAW264.7 macrophages were obtained from the ATCC (ATCC-TIB71).

In Vitro T-Cell Proliferation Assay

Primary CD4 T-cells specific to MOG35-55 were plated on round 96-well plates in medium containing RPMI-1640 supplemented with 2.5% fetal calf serum (FCS), 100 U/ml penicillin, 100 μg/ml streptomycin, 50 μM β-mercaptoethanol, and 2 mM L-glutamine. Each of the 96 wells had a final volume of 200 ul and contained 20×10³ T-cells, 5×10⁵ irradiated (25 gray) spleen cells (APC), with or without 1-5 μg/ml of MOG35-55. In addition, the relevant peptide was added. Each read was made with minimum of five repeats. In order to exclude interaction between the examined peptides and the MOG35-55 antigen, we initially added the MOG35-55 antigen to the APCs in a test tube, and in a second test tube we added the examined peptides to the T-cells. After 1 hour, we mixed the APCs with the T-cells and incubated them for 48 h in a 96 well round bottom plate. The T-cells were pulsed with 1 μCi (H³) thymidine, with a specific activity of 5.0 Ci/mmol, for 24 hours, and (H³) thymidine incorporation was measured using a 96-well plate beta-counter. The mean cpm±Standard Deviation (SD) was calculated for each quadruplicate or more. The results of T-cell proliferation experiments are shown as the percentage of T-cell proliferation inhibition triggered by the antigen in the absence of any peptide. The statistical analyses were performed using ANOVA. In several experiments, the T cells were directly activated with pre-coated CD3 and CD28 antibodies (LEAF™ purified anti-mouse clones 145-2-C11 and 37.51, respectively from Biolegend) at a final concentration of 2 μg/ml, or were activated with 50 ng/ml of Phorbol 12-myristate 13-acetate (PMA) together with 1 μM of ionomycine (Sigma Chemical Co., Israel).

Mice

C57BL/6J mice were kept in specific pathogen-free conditions. All mice were 2-3-month old when used in the experiments. The IACUC of the Weizmann Institute has approved the experiments, which were performed in accordance to its relevant guidelines and regulations.

XTT cytotoxicity assay

Aliquots of 2.5×10⁴ of the cells (from Jurkat E6-1, T-cell line) were distributed onto a 96-well plate (Falcon) in the presence of various peptide concentrations and incubated for 24 h. Wells in the last two columns served as blank (medium only), and 100% survival controls (cells and medium only), respectively. After incubation, the XTT reaction solution benzene sulfonic acid hydrate and N-methyl dibenzopyrazine methyl sulfate, mixed in a proportion of 50:1, was added for 2 more hr. Optical density was read at a 450-nm wavelength in an enzyme-linked immunoabsorbent assay plate reader. LC₅₀ (the concentration at which 50% of the cells die) was determined relative to the control, 2.5×10⁴ cells in medium. All assays were performed in duplicate.

Secondary Structure Determination by Using Circular Dichroism (CD) Spectroscopy

Circular dichroism (CD) spectroscopy measurements were performed by using an AppliedPhoto physics spectropolarimeter. The spectra were scanned using a thermostatic quartz cuvette with a path length of 1 mm. Wavelength scans were performed at 25° C.; the average recording time was 15 sec, in 1-nm steps, in the wavelength range of 190-260 nm. Peptides were scanned at a concentration of 10 μM in HEPES buffer (5 mM, pH 7.4).

Active Induction of Experimental Autoimmune Encephalomyelitis (EAE)

C57BL/6J mice were immunized subcutaneously at one site in the flank with 100 μL inoculum containing 200 μg antigen (MOG peptide; SEQ ID NO: 15) emulsified in CFA containing 300 μg Mycobacterium tuberculosis, with i.v. injection of pertussis toxin (300 ng/500 μL PBS) immediately and 48 h after the immunization. The mice were scored for clinical manifestations of EAE on a scale from 0 to 6. Disease onset typically occurred on days 11-12 after the encephalitogenic challenge. The gp41 derived peptide, pep 6, was dissolved in PBS, added to the inoculum emulsion and administered to the mice at day 0 of the immunization.

Ex Vivo Studies in Mice

Mice were subcutaneously immunized with 150 μg MOG35-55 emulsified in complete Freund's adjuvant (CFA) containing 150 μg Mt H37Ra with or without 0.5 mg/kg HIV peptides. Ten days post immunization, draining lymph nodes were removed and cultured in triplicate in the presence or absence of antigen, as previously described. The cultures were incubated for 72 h at 37° C. [3H] Thymidine (1 mCi/well) was added for an additional 16 h of incubation and the cultures were then harvested and counted using a Beta Counter. Pro-inflammatory cytokine analysis of IFNγ, IL-12, and TNFα were determined by ELISA, 24 h after cell activation, according to standard protocols from PharMingen (San Diego, Calif.), as described previously

Hemolysis of Human Red Blood Cells

Fresh human red blood cells (RBCs) were rinsed two times with PBS, followed by centrifugation for 7 min at 2000 rpm and resuspension in PBS. Peptides, at various concentrations, were dissolved in PBS and added to 50 μl of the stock hemolysis of human red blood cell (hRBC) solution in PBS for a final volume of 100 μl (final erythrocyte concentration, 4% (v/v)). The resulting suspension was incubated with agitation for 60 min at 37° C. The samples were then centrifuged at 2000 rpm for 7 min. The release of hemoglobin was monitored by measuring the absorbance of the supernatant at 540 nm. Controls for zero hemolysis (blank) and 100% hemolysis consisted of hRBCs suspended in PBS and PBS with 1% Triton, respectively.

Ex Vivo Studies in Mice

Mice were subcutaneously immunized with 150 μg MOG35-55 emulsified in complete Freund's adjuvant (CFA) containing 150 μg Mt H37Ra with or without 0.5 mg/kg HIV peptides. Ten days post immunization, draining lymph nodes were removed and cultured in triplicate in the presence or absence of antigen, as previously described. The cultures were incubated for 72 h at 37° C. [3H] Thymidine (1 mCi/well) was added for an additional 16 h of incubation and the cultures were then harvested and counted using a Beta Counter. Pro-inflammatory cytokine analysis of IFNγ, IL-12, and TNFα were determined by ELISA, 24 h after cell activation, according to standard protocols from PharMingen (San Diego, Calif.), as described previously.

Immunoprecipitation of Fluorescently Labeled Peptides with TCR

Jurkat T cells (4×10⁶) were incubated over night at 37° C. in the presence of 1 μM rhodamine-labeled peptide and lysed. The lysate was then incubated overnight with TCRα or GFP antibodies (2 μg) following 2 hours of incubation with protein G-plus Agarose beads (Santa Cruz Biotechnology Inc.). Next, the beads were washed with lysis buffer and boiled for 10 minutes; the protein supernatant was then run in a 12% SDS-PAGE. The presence of coimmunoprecipitated peptide was detected by the Typhoon 9400 variable mode imager.

Cellular Localization of Peptides in T Cells

Non-activated mMOG35-55 T cells (5×10⁴) were loaded with the lypophilic fluorescent dye, 1,1′-dioctadecyl-3,3,3′,3′,-tetramethylindodicarbocyanine, 4-chlorobenzenesulfonate salt (DiD) (Biotium, USA) or with the cytoplasmic fluorescent dye, 5- and 6-{[(4-chloromethyl)benzoyl]amino}tetramethylrhodamine (CMTMR) (Molecular Probes, USA). Jurkat T cells were also loaded with these dyes. Then the cells were washed, resuspended with culture medium, and activated for 24 hours. The NBD labeled fluorescent peptides (final concentration of 1 μM) were added to the cells for additional 1 hour incubation. The cells were washed to remove unbound peptides and were fixed with 4% paraformaldehyde for 20 min. Cells were then washed with PBS, deposited onto a glass slide, and cell samples were observed under a fluorescence confocal microscope (Olympus FV1000). NBD excitation was set at 488 nm, with the laser set at 10% power and fluorescence was recorded from 502-512 nm. CMTMR excitation was set at 559 nm, with the laser set at 2% power and fluorescence data were collected above 580 nm. Samples containing DiD labeling were excited at 635 nm, with the laser set at 2% power and fluorescence data were collected above 655 nm. Each cell was imaged for membrane or cytoplasmic staining (denoted in red) and for peptide staining (denoted in green). Using ImageJ, the images (n=20) were set to binary color and the two revised pictures were used to create a new picture of what only merged between them. The merged picture was divided by revised red picture to give the percentage of co-localization.

Confocal Setting for Co-Localization of Peptides with TCR Molecules

FITC excitation was set at 488 nm, with the laser set at 5% power and fluorescence was recorded from 502-525 nm. Rhodamine excitation was set at 559 nm, with the laser set at 1% power and the fluorescence data were collected above 580 nm. Each cell was imaged for TCRα staining (denoted in green) and for peptide staining (denoted in red). Using ImageJ, the images (n=20) were set to binary color and the two revised pictures were used to create a new picture of only what merged between them. The merged picture was divided by the revised red picture to give the percentage of co-localization.

Bioinformatics Analysis

A database was created from all the universal protein resource (UniProt) entries for a transmembrane protein of the envelope of HIV and SIV. These protein sequences were then run in a motif-based sequence analysis tool (MEME) that analyzes sequences for similarities among them and produces a description (motif) for each pattern it discovers. A motif that is short and unique in the protein was sought; therefore, the search was limited to a motif up to 10 residues long that is not repeated more than one time in the transmembrane protein. The motifs found were automatically arranged by their E-value, which is an estimate of the expected number of motifs with the given log likelihood ratio that one would find in a similarly sized set of random sequences. The log likelihood ratio is the logarithm of the ratio of the probability of the occurrences of the motif, given the motif model versus their probability, given the background model. The background model here is a 0-order Markov model using the background letter frequencies. The ‘Y’ axis is in BITS, which is equal to the relative entropy of the motif relative to a uniform background frequency model. The relative entropy of the motif, computed in bits and relative to the background letter frequencies, is equal to the log-likelihood ratio (llr) divided by the number of contributing sites of the motif times 1/ln(2). In order to better view the sequence, the MEME results plus the flanking regions were run through WebLogo, which is designed to generate sequence logos, as a method for graphical representation of amino or nucleic acid sequence alignment.

Fluorescent Measurements of Peptide-Membrane Interactions

Peptides interacting with membranes were analyzed and quantified using fluorescence anisotropy of their intrinsic Trp residue in the presence of large unilamellar vesicles (LUV) model membranes. The LUV were composed of phosphatidylcholine (PC) and cholesterol (Chol) (9:1) as previously described (Cohen et al. Biochemistry. 2008; 47(16):4826-4833). Excitation and emission wavelengths were set to 280/350 nm, respectively, and 1 μM of peptide (in 400 μl of PBS) was titrated with 13.3 mM membrane solution successively. Since Trp is known to change its emission in a hydrophobic environment, a change in its emission represents the amount of peptide bound to membranes. The system reached binding equilibrium (Fmax) at a certain lipid/peptide ratio, allowing us to calculate the affinity constant from the relations between the equilibrium level of Trp emission and the lipid concentration, using a steady-state affinity model. The affinity constants were then determined by non-linear least-squares (NLLSQ). The NLLSQ fitting was done using the following equation:

$$Y ( x ) = \frac{{K}_{a} \times X \times {F}_{m a x}}{1 + {K}_{a} \times X} \qquad ( 1 )$$

where X is the lipid concentration, Y(x) is the fluorescence emission, Fmax is the maximal difference in the emission of Trp-containing peptide before and after the addition of lipids (it represents the maximum amount of peptides bound to a lipid), and Ka is the affinity constant.

Fluorescent Measurements of NBD-Labeled Peptides

The fluorescence experiments were performed using NBD-labeled core peptides of TCRα (NBD-CP). Fluorescence spectra were obtained at room temperature, with excitation set at 467 nm (10 nm slit) and the emission scan at 500-600 nm (10 nm slits). In a typical experiment, the NBD-CP was first added from a stock solution in DMSO (final concentration of 0.1 μM and a maximum of 0.25% (v/v) DMSO) to a dispersion of PC:Chol LUV (200 μM) in PBS. This was followed by the addition of unlabeled HIV peptides in several sequential doses ranging from 2.5×10-3 μM to 0.4 μM. Fluorescence spectra were obtained before and after the addition of unlabeled HIV peptides. The fluorescence values were corrected by subtracting the corresponding blank (buffer with the same vesicle concentration).

Detection of Fluorescently Labeled Peptide Binding to T-Cell Proteins by SDS-PAGE

Jurkat T cells (4×10⁶) were incubated over night at 37° C. in the presence of 1 μM rhodamine-labeled pep6 (SEQ ID NO:3) and its mutant control peptide. Next, the cells were washed with PBS, crosslinked with 1% formaldehyde and after 10 minutes a 125 mM glycine solution was added to stop the reaction. Formaldehyde is a well known cross-linking agent used in the characterization of protein-protein interactions. The cells were lysed for 30 minutes on ice in a 300 μl lysis buffer followed by a sonication step. Insoluble material was removed by centrifugation at 3000×g for 4 min at 4° C. The proteins were resolved by 12% SDS-PAGE and proteins bound to the fluorescently labeled peptide were detected by the Typhoon 9400 variable mode imager (Amersham Biosciences). Excitation was set at 532 nm, and fluorescence emission was collected by a TAMRA filter (580 nm±30 nm) at 100 μm resolution and 600 volts. The protein loading amount was confirmed by Western blot to actin.

TNFα Secretion by LPS-Stimulated Macrophages

RAW264.7 Macrophages (2×10⁵ cells per well) were cultured overnight in a 96-well plate. The following day, the peptides (SEQ ID NO:3) were added to the cells at a final concentration of 1 μM. Cells were incubated with the peptide for 2 hours, then washed and incubated for 5 hours at 37° C. with fresh media containing 10 ng/ml of lipopolysaccharides (LPS) (Sigma Chemical Co., Israel), which was collected from each treatment. TNFα levels in each sample were evaluated using a mouse TNFα enzyme-linked immunosorbent assay kit (Biosource™ ELISA, Invitorgen) according to the manufacturer's protocol.

### Example 1

### Identification of a Highly Conserved Motif in the Transmembrane Ectodomains of HIV and SIV ENVs

Structure models of the HIV-1 gp41 core are available, though currently the structure of the gp41 loop region (comprising the N-terminal ISU and its possible C-terminal counterparts) had yet to be determined (Chan, et al., Cell, 1997. 89(2): p. 263-273; Buzon, V., et al., PLoS Pathog, 2010. 6(5): p. e1000880.). However, there is an NMR structure of the simian immunodeficiency virus (SIV) core together with the loop region (Caffrey et al., Embo J, 1998. 17(16): p. 4572-84). A computational aliment, based on both the NMR of the SIV and the similarity of the sequences between the envelope of SIV and HIV-1, provided a computational model for the core structure of the HIV-1 gp41 with the loop region (Caffrey, M., Biochim Biophys Acta, 2001. 1536(2-3): p. 116-22).

With the aim of finding HIV and SIV motifs that may modulate T-cell activation, a database was created from all reported transmembrane ENVs from HIV and SIV strains. Then, bioinformatic analysis was applied to search for a motif that is short and unique and with no repeats in the protein. The located motifs were automatically arranged by their E-value, which is an estimate of the expected number of motifs with the given log likelihood ratio that one would find in a similarly sized set of random sequences.

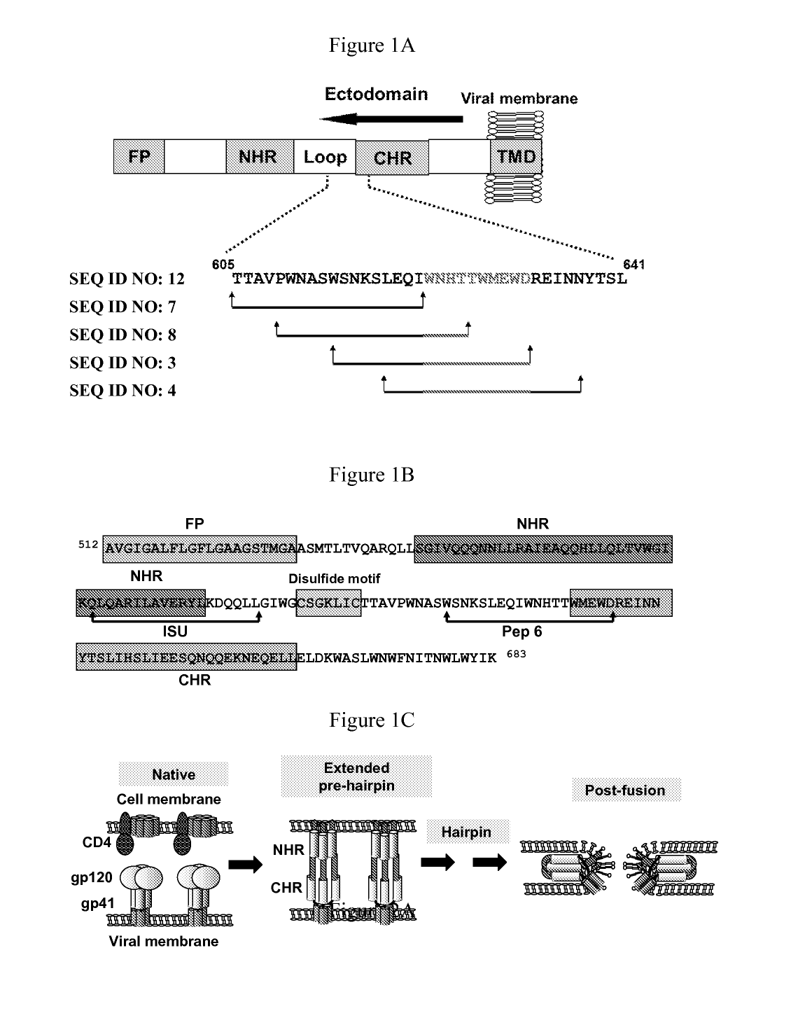

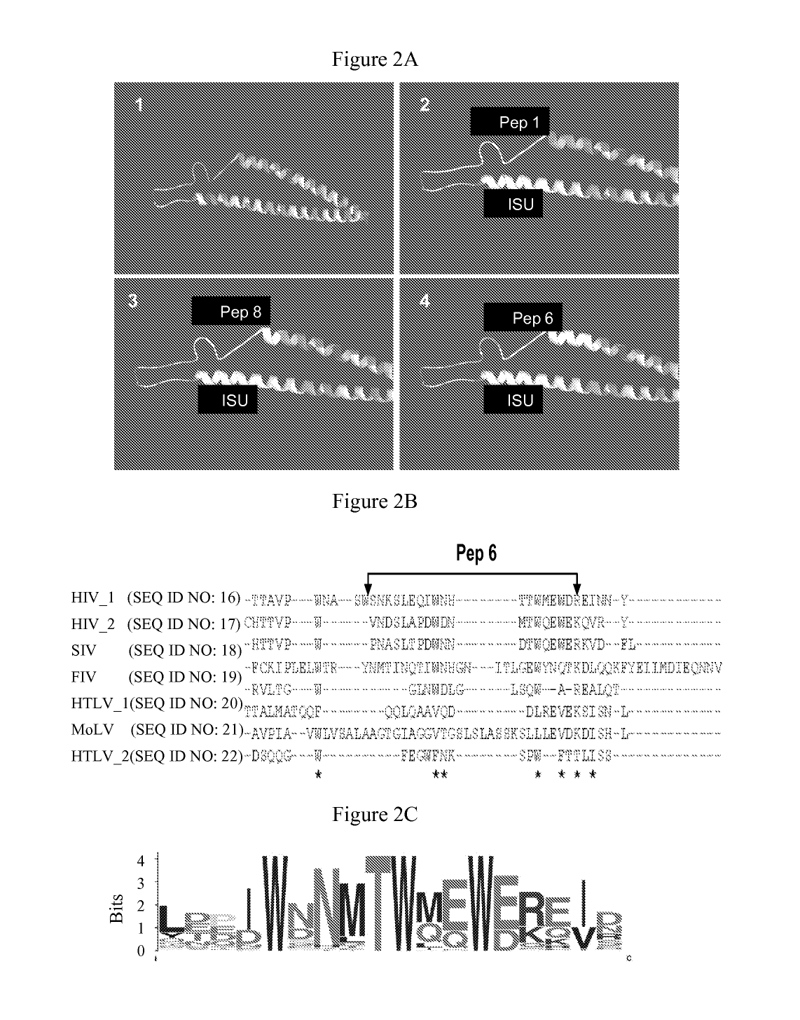

A computational model was used to propose candidates as the counterpart of the immunosuppressive unit (ISU) (FIG. 2A). In the HIV-1 loop model, there are 4 amino acids in the estimated region of the C-terminal counterpart that do not exist in the SIV. For this reason, the experiments focused on a relatively large sequence from that area (28 residues) in order to make sure the counterpart sequence was included. Sequences were screened within gp41 along the loop region toward the CHR region in order to cover residues from each side of the motif. This sequence was divided into three shorter fragments of 18 residues each (termed: pep 1 (SEQ ID NO: 7), pep 8 (SEQ ID NO: 8) and pep 6 (SEQ ID NO: 3)). Each sequence is shifted in its position with an overlapping of 8 residues to the previous sequence (Table 1 and FIG. 1A). The peptides were then tested for their ability to inhibit T-cell proliferation of primary T-cells (Example 2).

Interestingly, pep 6 (SEQ ID NO: 3) composed of a top-ranked motif, with an E-value of 1.1 e⁻⁵⁸⁶, based on 75 sites contributing to the construction of the motif, was found to be associated with the loop region of the ectodomain of the ENV transmembrane protein. The highly conserved motif was identified in various immunodeficiency viral strains (HIV-1, HIV-2, SIV) as well as leukemia viral strains (HTLV-1, HTLV-2, MoLV, FLV) (FIG. 2B). Notably, the motif consists of a repeat of three Trp residues that are adjacent to acidic residues (Glu and Asp) (FIG. 2C).

### Example 2

### Inhibition of Activated MOG35-55-Specific Line T Cells by Peptides Comprising the Highly Conserved Motif

To investigate the efficacy of different peptides comprising the highly conserved motif to inhibit the stimulation of antigen-specific line T cells, MOG35-55-specific murine line T cells (mMOG35-55 T-cells) were used. The preparation of the mMOG35-55 T-cells is detailed in the Methods section above. MOG35-55-specific line T cells were cultured in microtiter plates with irradiated syngeneic splenocytes as APCs and MOG35-55 in the presence or absence of several HIV gp41-derived peptides. Their proliferative response was measured in a H3-Thymidine proliferation assay.

The gp41 derived peptides exhibited dose-dependent inhibition of T-cell proliferation that relied on the location of the peptides with regard to the identified motif (FIGS. 3A and 3B). The order of potency was as follows: TTAVPWNASWSNKSLEQI (SEQ ID NO: 7) (not active)<WNASWSNKSLEQIWNHT (SEQ ID NO: 8)<SNKSLEQIWNHTTWMEWD (SEQ ID NO: 3) (most active)>EQIWNHTTWMEWDREINN (SEQ ID NO: 4).

The inhibitory concentration of the peptide of SEQ ID NO: 3 at 50% proliferation (IC₅₀) was 1.0 μM±0.3 μM (FIG. 3C), which was significantly more potent than the known immunosuppressive unit (ISU) of gp41 (FIG. 3D). In attempt to identify the specific element within the peptide that contributes to its potency, the IC₅₀ values of the various modified sequences were determined. The modified sequences and IC₅₀ values are specified in Table 1 below.

TABLE 1
Amino acid sequences and IC₅₀ values of
the examined peptides

| Name of | | SEQ | |
|---|---|---|---|
| the peptide | sequence | ID NO: | IC₅₀ (μM) |
| ISU | LQARILAVERYLKDQQL | 6 | 15 |
| PEP 1 | TTAVPWNASWSNKSLEQI | 7 | >40 |
| PEP 8 | WNASWSNKSLEQIWNHT | 8 | 15 |
| PEP 6 | SNKSLEQIWNHTTWMEWD | 3 | 0.55 |
| D-PEP 6 ^{a} | SNKSLEQIWNHTTWMEWD | 5 | 3 |
| SHORT PEP 6 | WNHTTWMEWD | 2 | 5 |
| (all L) | | | |
| SHIFTED PEP 6 | EQIWNHTTWMEWDREINN | 4 | 10 |

^{a} D amino acid incorporation to the sequence is bolded and underlined.

It was found that the peptides having the amino acid sequence of SEQ ID NO: 4 and of SEQ ID NO: 2 still exhibited potent activity, indicating that the WNHTTWMEWD sequence (SEQ ID NO: 2) is crucial for functionality. In addition, the incorporation of D-amino acids in the edges of the peptide preserved its activity.

Furthermore, T cells were cultured with APCs and MOG35-55 in the presence of 1 μM of the peptide of SEQ ID NO: 3. After 48 hr the media was collected and levels of IFNγ were detected by ELISA. As shown inf FIG. 3E, MOG35-55-stimulated T cells showed a reduction in IFNγ secretion when incubated with the peptide.

RAW264.7 macrophages incubated for 2 hr in the presence of 1 μM of peptide of SEQ ID NO:3 and then stimulated with LPS (10 ng/ml) for 5 hr. The media was collected and the levels of TNFα were detected by ELISA. FIG. 3F shows that the peptide did not inhibit TNFα secretion from lipopolysaccharides (LPS)-stimulated macrophages.

The viability of the cells was analyzed by an XTT cytotoxicity assay. The peptide was not toxic to T cells up to 20-fold more than the IC₅₀ of the peptide, the maximal concentration examined (FIG. 3G).

Hemolysis of red blood cells (RBCs) is often used to detect membranolytic activity of peptides. Hemolytic activity was measured by the release of hemoglobin into the media (OD 540 nm). Triton (1% v/v) served as a control for a hemolytic agent. Importantly, the peptide was not hemolytic up to 100-fold more than the IC₅₀ of the peptide, the maximal concentration examined (FIG. 3H).

The contribution of the motif within the peptide to its inhibitory activity was further investigated by either mutating the conserved Trp into Gly or by mutating the conserved acidic residues (Glu and Asp) into Gly (FIG. 3I). In both cases, a reduction in the peptide's inhibitory activity was observed (FIG. 3J).

Example 2 indicates that the peptide having the amino acid sequence of SNKSLEQIWNHTTWMEWD (SEQ ID NO: 3) has a specific inhibitory effect on T-cell proliferation. Further, the sequence of WNHTTWMEWD (SEQ ID NO: 2) is crucial for the T-cell proliferation inhibitory effect.

### Example 3

### Localization of the Gp-41 Derived Peptides with T-Cell Membranes

Analysis of the cellular localization of the gp-41 derived peptides of the invention in T cells may further suggest its plausible mode of action. For that purpose, human Jurkat T cells and mMOG35-55 T cells were probed with either fluorescent cytoplasmic dye (CMTMR) or with fluorescent membrane dye (DiD), which is known to accumulate in cell-membrane compartments. Fluorescently labeled NBD peptides were then added to the cells and were monitored for their cellular localization using confocal microscopy.

FIG. 4 shows the analysis of mMOG35-55 T cells (FIG. 4A) and Jurkat T cells (FIG. 4B). The left column shows the peptides' fluorescence, the central column shows cellular staining, and the right column shows a merged image between them. In both experiments a significant tendency was observed for the peptide of the invention to co-localize with membranes of murine and human T cells (FIG. 4C). Since Trp is known to change its emission in a hydrophobic environment, the binding affinity of the peptide to the membrane was determined by measuring the fluorescence anisotropy of the peptides' intrinsic Trp residue in the presence of the membrane (FIG. 4D). The solution of peptides was titrated with increasing concentrations of large unilamellar vesicles (LUVs) liposomes comprising phosphatidylcholine (PC) and cholesterol (Chol). The binding affinity constant was calculated as described in the Methods section above, yielding a value of 1.2×10⁴ M⁻¹ ±0.3×10⁴ M⁻¹.

Further, the T-cell binding affinity of peptides having the amino acid sequence of WNHTTWMEWD (SEQ ID NO:2 and 11; all L- and all D-amino acids, respectively) was examined. FIG. 4E shows that the fluorescently labeled peptides preferably bind T-cell lymphocytes derived from mice spleen cells. Spleen derived B cell lymphocytes were used as a control. Additionally, a control antimicrobial not-related peptide showed similar binding pattern to both lymphocyte populations.

Example 4 indicates that the gp-41 derived peptides of the invention, including those having the amino acid sequences of SEQ ID NO: 2, 3 and 11 localize with T-cell membranes with high binding affinity.

### Example 4

### The T-Cell Proliferative Signal Resulting from Antigen-Specific Stimulation by APCs is Inhibited by the Peptide's Interaction with the TCR

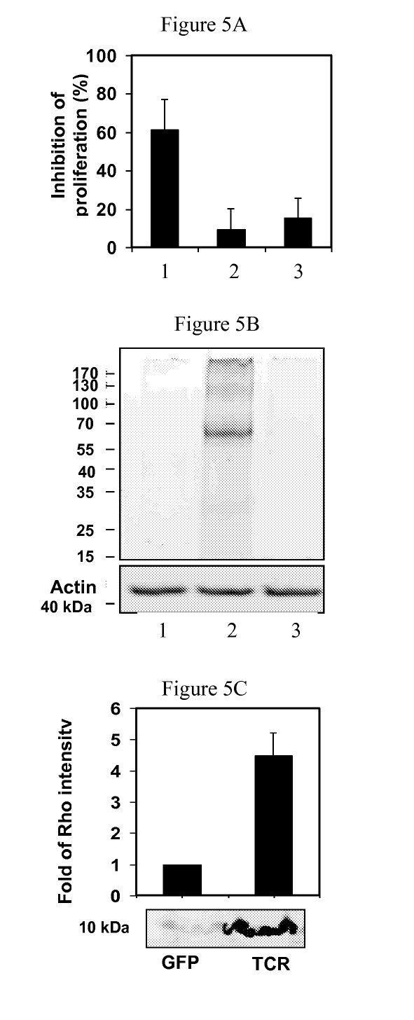

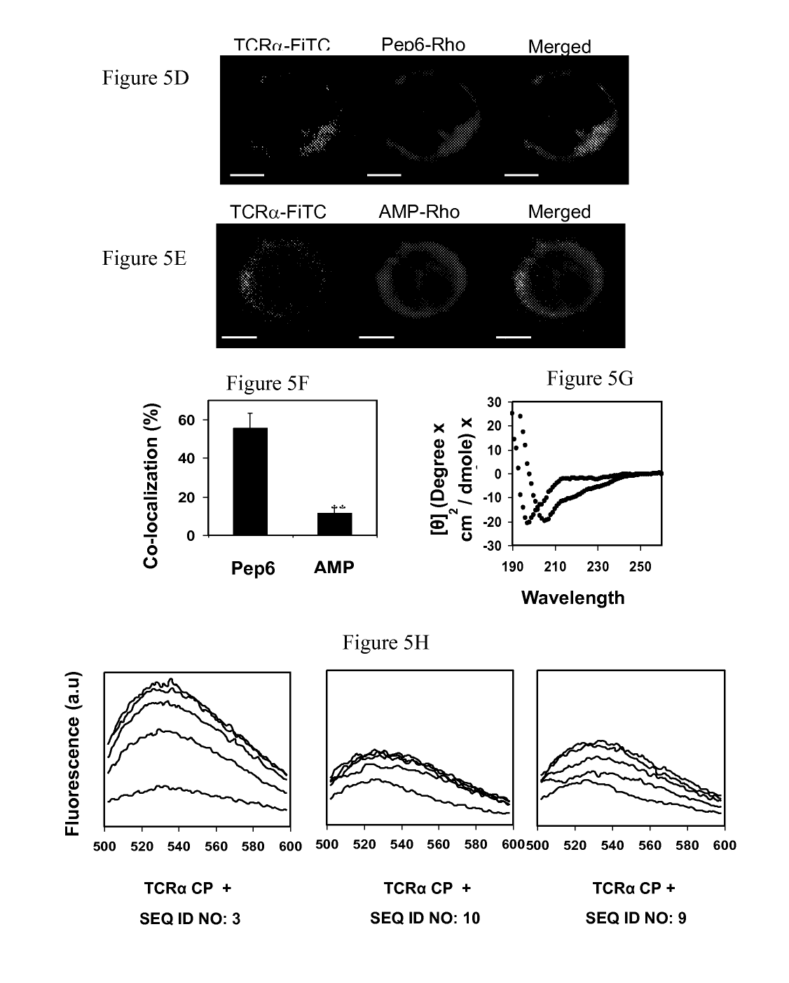

The inhibitory activity of the peptide was investigated by pinpointing the signaling step at which the peptide interfered. MOG35-55-specific line T cells were activated in the presence of pep 6 (SEQ ID NO: 3, 1 μM) by the following agents: (i) MOG35-55 antigen and APCs; (ii) CD3 and CD28 antibodies (2 μg/ml) and (iii) PMA (50 ng/ml) and ionomycine (1 μM). The uninhibited T-cell proliferative responses were 707±97 cpm and 14822±1541 cpm for CD3/CD28 antibodies and PMA/ionomycine, respectively. The background proliferation levels in the absence of CD3/CD28 antibodies and PMA/ionomycine were 66±6 cpm and 105±17 cpm, respectively. As shown in FIG. 5A the peptide inhibited T-cell proliferative signals that result from cognitive TCR engagement through antigen presenting cells (APCs). Nevertheless, the peptide did not inhibit anti-CD3/CD28 or PMA/ionomycine stimulated T-cell proliferation. This suggests that the peptide might interact with the components of the TCR complex to achieve its inhibitory effect.

To investigate the interaction of the peptide with the TCR, 1 μM fluorescently labeled Rho-peptides were incubated with Jurkat T cells and then cross-linked with formaldehyde to maintain the interaction with their target proteins. The cross-linked T-cell lysate was resolved by SDS-PAGE and proteins bound to Rho-peptides were detected by the fluorescence of rhodamine (FIG. 5B). Remarkably, only one major fluorescent band was detected at the protein size of ˜70 kDa that could match the stoichiometry corresponding to the TCR heterodimer. Fluorescent bands were neither observed in the T-cell lysates with no peptides added nor with the addition of the control mutant (W/G) peptide (SNKSLEQIGNHTTGMEGD as set forth in SEQ ID NO: 9). Western blot for actin was used to assure equal loading. Immunoprecipitation using antibodies against the TCR confirmed that the labeled Rho-peptide interacts with the TCR. As a control, a non-related GFP antibody was used. Further, Jurkat T cells were incubated with Rho-pep 6 (SEQ ID NO: 3), lysed, and immunoprecipitated with antibodies to TCRα or GFP. Bound proteins were separated by SDS-PAGE and analyzed for the presence of the fluorescently-labeled peptides. As seen in FIG. 5C the Rho-peptide coprecipitated with the TCR.

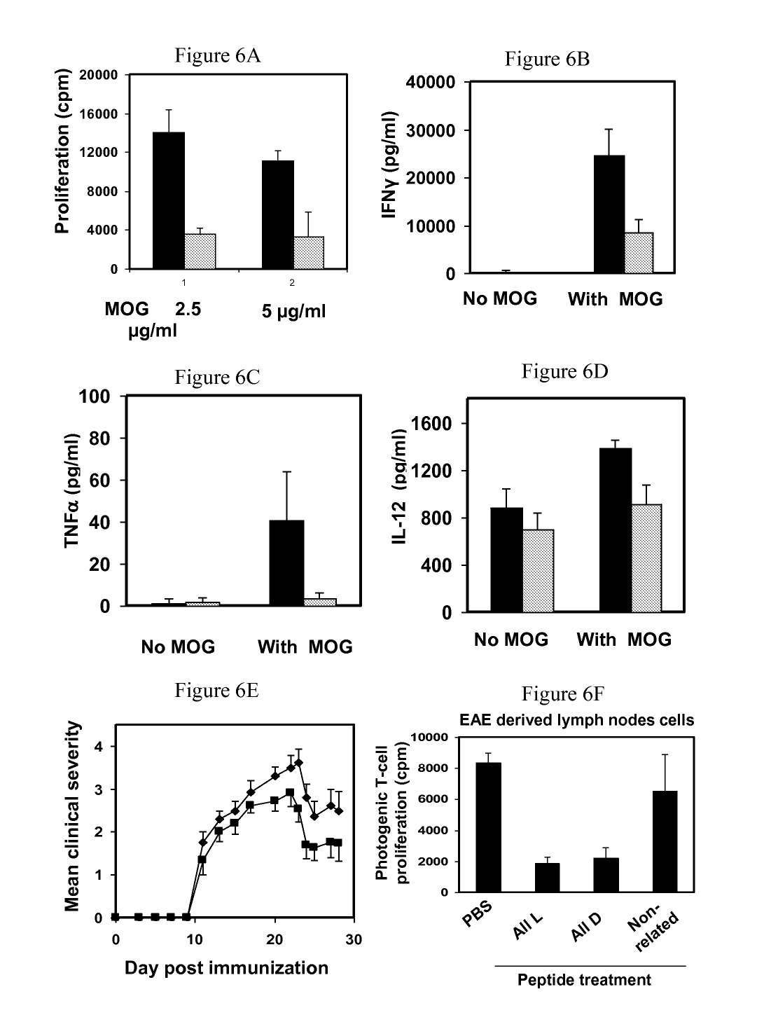

Co-localization of the peptide with the TCR was assessed utilizing confocal microscopy (FIG. 5 D-E). Antigen-stimulated mMOG35-55-specific line T cells were probed with antibodies against TCRα followed by staining with secondary FITC-labeled antibodies (left column) and with the Rho-labeled peptide (SEQ ID NO: 3) (middle column). To evaluate the co-localization between the molecules, a merged image was produced (right column). FIG. 5D shows a capping shape of the TCRα molecules in activated T cells, which were co-localized with a similar distribution shape of the peptide. As a control, AMP (SEQ ID NO: 13), a non-related antimicrobial peptide (Papo et al. J Biol Chem. 2005; 280(11):10378-10387), was labeled with rhodamine and its localization on T cells was detected. Unlike Rho-peptide, Rho-AMP was found throughout the cell membrane (FIG. 6E). The co-localization percentages of the peptides with the TCRα molecules were significantly higher in the peptide experiment than the ones displayed in the AMP experiment (FIG. 6F).

The TCR complex's TMD helices mediate receptor activation through the interactions between basic and acidic residues. Thus it is reasonable that the membrane-bound acidic peptide of the invention would favor its interaction with the TMD of the TCR. Moreover, the peptide exhibited an α-helical structure in the lipid environment (FIG. 5G) which might facilitate its interaction with other transmembrane helices. To investigate this possibility, we utilized NBD fluorescence, which is sensitive to the membrane surroundings; its emission intensity is increased via membrane insertion (Rapaport et al. Embo J. 1995; 14(22):5524-5531). Thus, it enables one to track the membrane-bound state of a marked protein fragment. The core peptides of TMD from TCRα, termed CP (GLRILLLKV; SEQ ID NO: 14), were labeled with NBD. Their initial fluorescence emission spectra were first measured in the presence of LUVs alone and then in the presence of several sequential doses of unlabeled peptide and its mutants (SEQ ID NO: 3, 9 and 10) (FIG. 5H). NBD-CP exhibited low-fluorescence signals in the presence of liposomes alone. However, when the unlabeled peptide was added, the fluorescence emission maxima of NBD-CP increased sharply, concomitant with a blue shift. Notably, when the peptide's mutants (SEQ ID NO: 9 and 10) were added, only a slight increase was observed in the emission maxima of NBD-CP (FIG. 5H).

Example 4 indicates that the gp41 derived peptide specifically recognizes CP, which leads to changes in its environment probably by penetrating into the membrane.

### Example 5

### Effect of Gp-41 Derived Peptide (SEQ ID NO: 3, 2 and 11) on MOG35-55-Induced Experimental Autoimmune Encephalomyelitis in Mice

The finding that gp41 derived peptides (SEQ ID NO: 3) inhibit mMOG35-55 T cells in vitro suggests that it might also inhibit pathogenic MOG35-55-specific T cells in vivo. Therefore, experiments were carried out aimed at investigating the effect of the peptide on experimental autoimmune encephalomyelitis (EAE), an animal model of Multiple Sclerosis. Multiple Sclerosis is an inflammatory autoimmune disease of the central nervous system (CNS) characterized by neurological impairment, resulting from primary demyelination and axonal damage. The pathogenic mechanism underlying disease development involves CNS-specific T-cell activation and Th1 differentiation, followed by infiltration of T cells, B cells, and macrophages into the CNS.

First, the toxicity of the peptide was investigated by injecting naïve C57BL mice i.v. with 50 mg/kg of the peptides. No toxic effect was observed in these mice (n=3). Then, C57Bl/6J mice that were induced to develop clinical EAE by immunization with MOG35-55/CFA were given a dose of the peptide that was 100-fold less than the one examined in the toxicity assay, i.e. 10 μg per mouse (0.5 mg/kg), or PBS as a control. On day 10 post immunization, prior to the onset of clinical signs of EAE, the lymph node cells (LNCs) from each treatment group were cultured for 72 h in microtiter wells in triplicate (0.5×10⁶/well) in the absence or presence of MOG35-55 (2.5 or 5 μg/ml; SEQ ID NO: 15). [H3] Thymidine was added for the last 18 h. The LNCs were analyzed ex vivo for their recall proliferative response to the immunizing MOG35-55 peptide and for secretion of pro-inflammatory cytokines. As shown in FIG. 6A, LNCs from mice that were treated with the peptide exhibited a significant reduction in their proliferative response to the immunizing MOG35-55 peptide, compared with LNCs from mice treated with PBS.

Given that the inhibition of the T-cell proliferative response by the peptide was associated with reduction in pro-inflammatory cytokine secretion in vitro (FIG. 3C); the corresponding effect was examined ex vivo. LNCs, derived from PBS-treated mice stimulated with the immunizing MOG35-55 peptide (5 μg/ml, 24 h), showed higher levels of IFNγ secretion (25 ng/ml) compared with LNCs derived from peptide-treated mice (9 ng/ml) (64% inhibition; FIG. 6B). Similarly, while LNCs derived from PBS-treated mice stimulated with the MOG35-55 peptide, secreted 40 pg/ml TNFα; the secretion of TNFα by LNCs derived from SEQ ID NO:3-treated mice was barely detected upon stimulation by MOG35-55 (90% inhibition; FIG. 6C).

IL-12 is a cytokine mostly produced by antigen-presenting cells. Primed LNCs (containing T, B, and antigen presenting cells) from PBS or peptide-treated mice, showed a comparable significant basal level of secreted IL-12 without stimulation by MOG35-55 (FIG. 6D). In LNCs from PBS-treated mice as opposed to LNCs from peptide-treated mice, ex vivo stimulation by the MOG35-55 peptide resulted in a slight, yet significant, increase in IL-12 secretion (FIG. 6D). This does not necessarily mean suppression of IL-12 secretion by the peptide. Rather, the slight increase in IL-12 secretion by PBS-treated mice compared with peptide-treated mice LNCs is more likely to be attributed to the higher level of IFNγ secreted by stimulated MOG35-55-specific T cells (FIG. 6B). By a positive feedback mechanism, this may in turn promote IL-12 production in antigen presenting cells.

Subsequently, the inventors examined the onset of the clinical manifestation of the EAE, which started on day 10 after the encephalitogenic challenge both in PBS and in peptide-treated mice (FIG. 6E). Importantly, from day 20, a significant reduction in EAE severity was observed in the peptide-treated group. The reduction in disease severity was found to result from down-regulation of the encephalitogenic MOG35-55 reactive T cells by the peptide SEQ ID NO:3 (FIG. 6 A-D). This result demonstrates the ability of the peptides of the invention to modulate antigen-specific T-cell activation in vivo.

FIG. 6F shows ex-vivo down-regulation of encephalitogenic reactive T cells following administration of the short gp-41 derived peptides (SEQ ID NO:2 and 11; all L- and all D-amino acids, respectively) (0.5 mg/kg˜10 μg per mouse). C57Bl mice were injected for EAE induction. Ten days after immunization, draining lymph node cells from each treatment group were pooled and analyzed for their ex vivo recall proliferative response to MOG35-55. As a control, a non-related peptide was used

Overall, Example 5 shows that administration of the gp-41 derived peptides suppresses MOG35-55-induced experimental autoimmune encephalomyelitis in mice by down-regulation of pathogenic T cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An isolated peptide of 10-30 amino acids comprising the amino acid sequence WNHTTWMEWD as set forth in SEQ ID NO: 2, or an analog thereof comprising at least one D amino acid.

2. The isolated peptide of claim 1, comprising an amino acid sequence selected from the group consisting of:
SNKSLEQIWNHTTWMEWD (SEQ ID NO: 3),
EQIWNHTTWMEWDREINN (SEQ ID NO: 4), and
SNKSLEQIWNHTTWMEWD, wherein S is D-Ser and D is D-Asp (SEQ ID NO: 5).

3. An isolated peptide of 8-30 amino acids comprising the amino acid sequence HTTWMEWD as set forth in SEQ ID NO: 1, or an analog or a salt thereof, wherein the analog is selected from an analog comprising at least on D amino acid or an analog comprising a substitution of at least one Trp (W) residue with an amino acid selected from Ile (I), Leu (L) and Gly (G).

4. The isolated peptide of claim 3, comprising an amino acid sequence selected from the group consisting of:
WNHTTWMEWD (SEQ ID NO: 2),
SNKSLEQIWNHTTWMEWD (SEQ ID NO: 3), and
EQIWNHTTWMEWDREINN (SEQ ID NO: 4).

5. The isolated peptide of claim 3, consisting of the amino acid sequence HTTWMEWD (SEQ ID NO: 1).

6. The isolated peptide of claim 3, consisting of the amino acid sequence WNHTTWMEWD (SEQ ID NO: 2).

7. The isolated peptide of claim 3, consisting of the amino acid sequence SNKSLEQIWNHTTWMEWD (SEQ ID NO: 3).

8. The isolated peptide of claim 3, consisting of the amino acid sequence EQIWNHTTWMEWDREINN (SEQ ID NO: 4).

9. The isolated peptide of claim 3, wherein the at least one D amino acid is in a position selected from the peptide's N-terminus, C-terminus or both.

10. The isolated peptide of claim 9, consisting of the amino acid sequence SNKSLEQIWNHTTWMEWD (SEQ ID NO: 5), wherein S is D-Ser and D is D-Asp.

11. The isolated peptide of claim 3, consisting of the amino acid sequence WNHTTWMEWD (SEQ ID NO: 11), wherein all amino acids are D-amino acids.

12. A pharmaceutical composition comprising as an active ingredient the isolated peptide according to claim 1, and a pharmaceutically acceptable carrier.

13. The pharmaceutical composition of claim 12, further comprising an immunosuppressive agent.

14. The pharmaceutical composition of claim 13, wherein the immunosuppressive agent is an immunosuppressive peptide.

15. The pharmaceutical composition of claim 14, wherein the immunosuppressive peptide comprises an amino acid sequence LQARILAVERYLKDQQL as set forth in SEQ ID NO: 6, or an analog, derivative or a salt thereof.

16. A method of treating a T cell mediated disease or disorder in a subject in need thereof, comprising administering to the subject a therapeutically effective amount of the pharmaceutical composition according to claim 12.

17. The method of claim 16, wherein the T cell mediated disease or disorder is a T cell-mediated autoimmune disease.

18. The method of claim 17, wherein the T cell-mediated autoimmune disease is selected from the group consisting of: multiple sclerosis, autoimmune neuritis, systemic lupus erythematosus (SLE), psoriasis, Type I diabetes (IDDM), Sjogren's disease, thyroid disease, myasthenia gravis, sarcoidosis, autoimmune uveitis, inflammatory bowel disease (Crohn's and ulcerative colitis), autoimmune hepatitis, rheumatoid arthritis, idiopathic thrombocytopenia, scleroderma, alopecia areata, glomerulonephritis, dermatitis and pemphigus.

19. The method of claim 17, wherein the autoimmune disease is multiple sclerosis.

20. The method of claim 16, wherein the T cell mediated disease or disorder is a T cell-mediated inflammatory disease.

21. The method of claim 20, wherein the T cell mediated disease or disorder is selected from the group consisting of: allograft rejection and graft-versus-host disease.


SEQUENCE LISTING (23 sequences)

SEQ ID NO: 1 - protein, 8 aa, Artificial Sequence
  Synthetic peptide
HTTWMEWD

SEQ ID NO: 2 - protein, 10 aa, Artificial Sequence
  Synthetic peptide
WNHTTWMEWD

SEQ ID NO: 3 - protein, 18 aa, Artificial Sequence
  Synthetic peptide
SNKSLEQIWNHTTWMEWD

SEQ ID NO: 4 - protein, 18 aa, Artificial Sequence
  Synthetic peptide
EQIWNHTTWMEWDREINN

SEQ ID NO: 5 - protein, 18 aa, Artificial Sequence
  Synthetic peptide
SNKSLEQIWNHTTWMEWD

SEQ ID NO: 6 - protein, 17 aa, Artificial Sequence
  Synthetic peptide
LQARILAVERYLKDQQL

SEQ ID NO: 7 - protein, 18 aa, Artificial Sequence
  Synthetic peptide
TTAVPWNASWSNKSLEQI

SEQ ID NO: 8 - protein, 17 aa, Artificial Sequence
  Synthetic peptide
WNASWSNKSLEQIWNHT

SEQ ID NO: 9 - protein, 18 aa, Artificial Sequence
  Synthetic peptide
SNKSLEQIGNHTTGMEGD

SEQ ID NO: 10 - protein, 18 aa, Artificial Sequence
  Synthetic peptide
SNKSLEQIWNHTTWMGWG

SEQ ID NO: 11 - protein, 10 aa, Artificial Sequence
  Synthetic peptide
WNHTTWMEWD

SEQ ID NO: 12 - protein, 37 aa, Human immunodeficiency virus type 1
TTAVPWNASWSNKSLEQIWNHTTWMEWDREINNYTSL

SEQ ID NO: 13 - protein, 15 aa, Artificial Sequence
  Synthetic peptide
KLKLLKLLKLLKLLK

SEQ ID NO: 14 - protein, 9 aa, Artificial Sequence
  Synthetic peptide
GLRILLLKV

SEQ ID NO: 15 - protein, 21 aa, Artificial Sequence
  Synthetic peptide
MEVGWYRSPFSRVVHLYRNGK

SEQ ID NO: 16 - protein, 34 aa, Human immunodeficiency virus type 1
TTAVPWNASWSNKSLEQIWNHTTWMEWDREINNY

SEQ ID NO: 17 - protein, 30 aa, Human immunodeficiency virus type 2
CHTTVPWVNDSLAPDWDNMTWQEWEKQVRY

SEQ ID NO: 18 - protein, 30 aa, Simian immunodeficiency virus
HTTVPWPNASLTPDWNNDTWQEWERKVDFL

SEQ ID NO: 19 - protein, 54 aa, Feline immunodeficiency virus
FCKIPLELWTRYNMTINQTIWNHGNITLGEWYNQTKDLQQKFYEIIMDIEQNNV

SEQ ID NO: 20 - protein, 24 aa, Human T-cell lymphotropic virus type 1
RVLTGWGLNWDLGLSQWAREALQT

SEQ ID NO: 21 - protein, 31 aa, Moloney murine sarcoma virus
TTALMATQQFQQLQAAVQDDLREVEKSISNL

SEQ ID NO: 22 - protein, 45 aa, Human T-cell lymphotropic virus type 2
AVPIAVWLVSALAAGTGIAGGVTGSLSLASSKSLLLEVDKDISHL

SEQ ID NO: 23 - protein, 23 aa, Feline leukemia virus
DSQQGWFEGWFNKSPWFTTLISS